(19) United States
(12) Patent Application Publication
(10) Pub. No.: US 2018/0230539 A1
(43) Pub. Date: Aug. 16, 2018

(54) CELL-FREE METHYLATED AND UNMETHYLATED DNA IN DISEASES RESULTING FROM ABNORMALITIES IN BLOOD GLUCOSE LEVELS

(71) Applicants: Indiana University Research and Technology Corporation, Indianapolis, IN (US); Universite Libre De Bruxelles, Brussels (BE)

(72) Inventors: Raghavendra G. Mirmira, Zionsville, IN (US); Sarah A. Tersey, Indianapolis, IN (US); Decio Laks Eizirik, Brussels (BE); Francois Fuks, Brussels (BE)

(21) Appl. No.: 15/746,262

(22) PCT Filed: Jul. 21, 2016
(86) PCT No.: PCT/US16/43407

§ 371 Date: Jan. 19, 2018

Related U.S. Application Data
(60) Provisional application No. 62/328,817, filed on Apr. 28, 2016.
(60) Provisional application No. 62/291,018, filed on Feb. 4, 2016.
(60) Provisional application No. 62/194,936, filed on Jul. 21, 2015.

(51) Int. Cl. C12Q 1/6883

(52) U.S. Cl. CPC C12Q 1/6883; C12Q 2600/112; C12Q 2600/154

(57) ABSTRACT

Disclosed herein are compositions and methods for determining new-onset type 1 diabetes, methylation-specific polymerase chain reaction assays for determining new-onset type 1 diabetes, methods for distinguishing between type 1 diabetes and type 2 diabetes, methods for dysglycemia in obese adolescent subjects including obese adolescent subjects having type 2 diabetes and methylation-specific polymerase chain reaction assays for determining dysglycemia in obese adolescent subjects including obese adolescent subjects having type 2 diabetes.

### CROSS-REFERENCE TO RELATED APPLICATIONS

This application claims priority to U.S. Provisional Patent Application Nos. 62/194,936, filed on Jul. 21, 2015, 62/291,018, filed on Feb. 4, 2016, and 62/328,817, filed on April 28, 2016, each of which is incorporated by reference herein in its entirety.

### STATEMENT OF GOVERNMENT SUPPORT

This invention was made with government support under DK104166 awarded by the National Institutes of Health. The U.S. Government has certain rights in the invention.

### STATEMENT IN SUPPORT FOR FILING A SEQUENCE LISTING

A paper copy of the Sequence Listing and a computer readable form of the Sequence Listing containing the file named “IURTC_2015-150-02_ST25.txt”, which is 8,658 bytes in size (as measured in MICROSOFT WINDOWS® EXPLORER), are provided herein and are herein incorporated by reference. This Sequence Listing consists of SEQ ID NOs:1-9.

### BACKGROUND OF DISCLOSURE

The present disclosure relates to unmethylated and methylated DNA as biomarkers of diseases resulting from abnormalities in blood glucose levels and indicators of disease progression. More particularly, the present disclosure relates to unmethylated and methylated preproinsulin (INS) DNA as biomarkers of β cell death in new-onset of type 1 diabetes, in impaired glucose tolerance, in type 2 diabetes, in obese adolescents with impaired glucose tolerance, and as indicators of disease progression.

Although much attention is focused on the rising epidemic of type 2 diabetes, type 1 diabetes (T1D) has recently seen an inexplicable increase in incidence and prevalence in youth. The development of type 1 diabetes is progressive, with early islet inflammation followed by loss of pancreatic β cell function and mass. Particularly, T1D is an autoimmune disorder in which loss of tolerance causes the targeted destruction of insulin-producing islet β cells. Further, development of hyperglycemia extends over a period that spans years.

The diagnosis of T1D has conventionally been based on blood glucose criteria and is typically identified at a time when individuals have lost substantial β cell mass and function. Interventions instituted at the time of T1D diagnosis have uniformly failed to recover β cell mass and function, raising the possibility that detection of β cell death earlier in the course of disease (prior to hyperglycemia) might provide an opportunity for earlier institution of therapy. Immunologic biomarkers (e.g. autoantibodies) offer the ability to identify those at increased risk of developing T1D, but use of such criteria alone still does not predict with certainty those who will ultimately develop T1D.

Recently, the measurement of circulating unmethylated DNA encoding preproinsulin (INS) has been proposed as a biomarker of β cell death. Particularly, it was previously shown that INS DNA in β cells has a much higher frequency of unmethylated CpG sites compared to other cell types. Further, the relative abundance of unmethylated INS DNA in the circulation was shown to be elevated in both mice and humans with recent-onset T1D, and higher relative abundances correlated temporally to more active β cell destruction. In these previous studies, unmethylated INS DNA was expressed as a ratio relative to methylated INS DNA as a method for normalization, since methylated INS DNA is assumed to remain constant and independent of the underlying disease process. However, because β cells and many other cell types in the islet contain some fraction of both unmethylated and methylated INS DNA, it remains unclear to what extent each species of INS DNA might be independently informative of the underlying process in diseases resulting from abnormalities in blood glucose levels.

During obesity, insulin resistance in the muscle, liver, and adipose tissue increases the demand for insulin secretion to maintain glucose homeostasis, resulting in an increased demand for the β cells to compensate and hypersecrete insulin (Martin et al. 1992). This adaptation is characterized by maintenance of normal glucose levels and normal β cell gene expression through an increase in insulin secretion and β cell mass (Steil et al. 2001). The inability of the β cell to fully compensate leads to hyperglycemia, and further deficiencies in β cell mass and function are major determinants in the transition from glucose intolerance to overt type 2 diabetes (T2D) (Prentki and Nolan 2006). The lack of functional β cells could occur due to the lack of compensation, apoptosis and cell death, and/or dedifferentiation of the β cell. Previous findings in cadaveric human pancreata show an increase in apoptotic β cell number in T2D in comparison to individuals that had normal glucose tolerance (NGT) (Butler et al. 2003). Furthermore, recent findings suggest that dedifferentiation of the β cell to a progenitor-like state may also be the cause of β cell mass loss during the development of T2D (Talchai et al. 2012). However, procedures to detect the activity of these mechanisms in vivo have yet to be established.

In obese youth, β cell function worsens with increasing dysglycemia, but it is unknown if β cell death is coincident with this worsening in function. Elevations in circulating, cell-free methylated and unmethylated preproinsulin (INS) DNA in youth with new-onset T1D indicate that β cell death is detectable at T1D diagnosis with this measurement.

Accordingly, there is a need for biomarkers and diagnostic methods for evaluating biomarkers that are specific for diseases resulting from abnormalities in blood glucose levels such as T1D, T2D, dysglycemia, and impaired glucose tolerance. It would further be advantageous if the biomarkers could be used to determine the stages of disease progression.

### BRIEF DESCRIPTION

The present disclosure is generally related to evaluating circulating methylated and unmethylated DNA that are not only specific for diseases resulting from abnormalities in blood glucose levels, but are also relevant to different stages of disease progression. Particularly, it has been unexpectedly found that both unmethylated and methylated INS DNA levels are independently and specifically altered in T1D. The present disclosure is further related to evaluating circulating methylated and unmethylated INS DNA that are not only specific for T2D, but are also relevant to different stages of disease progression. Particularly, it has been unexpectedly found that both unmethylated and methylated INS DNA levels are independently and specifically altered in T2D. The present disclosure is also related to evaluating circulating methylated and unmethylated INS DNA that are not only specific for dysglycemia in obese adolescents, but are also relevant to different stages of disease progression. Particularly, it has been unexpectedly found that both unmethylated and methylated INS DNA levels are independently and specifically altered in dysglycemia in obese adolescents. The present disclosure is also directed to probes and methods for methylation-specific polymerase chain reaction assays.

Accordingly, in one aspect, the present disclosure is directed to use of a circulating unmethylated DNA, a circulating methylated DNA, and combinations thereof as a biomarker for one or more of the group consisting of Type 1 diabetes, new-onset Type 1 diabetes, Type 2 diabetes, impaired glucose tolerance, and dysglycemia.

In another aspect, the present disclosure is directed to use of an oligonucleotide of SEQ ID NO:3 in the determination of a methylated preproinsulin DNA in a sample.

In another aspect, the present disclosure is directed to use of an oligonucleotide of SEQ ID NO:4 in the determination of an unmethylated preproinsulin DNA in a sample.

In another aspect, the present disclosure is directed to a diagnostic kit comprising one or more of the group consisting of an oligonucleotide of SEQ ID NO:1, an oligonucleotide of SEQ ID NO:2, an oligonucleotide of SEQ ID NO:3, and an oligonucleotide of SEQ ID NO:4.

In another aspect, the present disclosure is directed to an assay device comprising one or more of the group consisting of an oligonucleotide of SEQ ID NO:1, an oligonucleotide of SEQ ID NO:2, an oligonucleotide of SEQ ID NO:3, and an oligonucleotide of SEQ ID NO:4.

In another aspect, the present disclosure is directed to an oligonucleotide probe comprising SEQ ID NO:3 and a label.

In another aspect, the present disclosure is directed to an oligonucleotide probe comprising SEQ ID NO:4 and a label.

In another aspect, the present disclosure is directed to a primer pair comprising a first oligonucleotide comprising SEQ ID NO:3 and a label and a second oligonucleotide comprising SEQ ID NO:4 and a label.

In another aspect, the present disclosure is directed to a method for determining new-onset type 1 diabetes in a subject suspected of having new-onset type 1 diabetes. The method comprises: amplifying methylated preproinsulin (INS) DNA in a sample obtained from the subject suspected of having new-onset type 1 diabetes; amplifying unmethylated preproinsulin (INS) DNA in the sample obtained from the subject suspected of having new-onset type 1 diabetes; detecting whether a nucleotide located at position −69 from the preproinsulin (INS) transcriptional start site is methylated or unmethylated; comparing the concentration of methylated preproinsulin (INS) DNA and unmethylated preproinsulin (INS) DNA in the sample with the concentration of methylated preproinsulin (INS) DNA and unmethylated preproinsulin (INS) DNA in a control; and determining that the subject has new-onset type 1 diabetes when the concentration of methylated preproinsulin (INS) DNA and unmethylated preproinsulin (INS) DNA in the sample is greater than the concentration of methylated preproinsulin (INS) DNA and unmethylated preproinsulin (INS) DNA in the control.

In another aspect, the present disclosure is directed to a methylation-specific polymerase chain reaction assay for determining new-onset type 1 diabetes in a subject suspected of having new-onset type 1 diabetes. The method comprises: isolating DNA from a sample; treating the isolated DNA with bisulfite to convert unmethylated cytosine to uracil; amplifying unmethylated preproinsulin (INS) DNA in a sample obtained from the subject suspected of having new-onset type 1 diabetes; amplifying methylated preproinsulin (INS) DNA in the sample obtained from the subject suspected of having new-onset type 1 diabetes; and detecting whether a nucleotide located at position −69 from the preproinsulin (INS) transcriptional start site is methylated or unmethylated.

In another aspect, the present disclosure is directed to a method for distinguishing between type 1 diabetes and type 2 diabetes in a subject suspected of having type 1 diabetes or type 2 diabetes. The method comprises: amplifying methylated preproinsulin (INS) DNA in a sample obtained from the subject suspected of having type 1 diabetes; amplifying unmethylated preproinsulin (INS) DNA in the sample obtained from the subject suspected of having new-onset type 1 diabetes; detecting whether a nucleotide located at position −69 from the preproinsulin (INS) transcriptional start site is methylated or unmethylated; comparing the concentration of methylated preproinsulin (INS) DNA and the concentration of unmethylated preproinsulin (INS) DNA from the subject to the concentration of methylated preproinsulin (INS) DNA and the concentration of unmethylated preproinsulin (INS) DNA from a subject having type 2 diabetes; and diagnosing the subject as being suspected of having type 1 diabetes if the concentration of methylated preproinsulin (INS) DNA and the concentration of unmethylated preproinsulin (INS) DNA from the subject are elevated when compared to the concentration of methylated preproinsulin (INS) DNA and the concentration of unmethylated preproinsulin (INS) DNA of the subject having type 2 diabetes.

In another aspect, the present disclosure is directed to a method for diagnosing new-onset type 1 diabetes in a subject suspected of having new-onset type 1 diabetes. The method comprises: amplifying methylated biomarker DNA in a sample obtained from the subject suspected of having new-onset type 1 diabetes; amplifying unmethylated biomarker DNA in thesample obtained from the subject suspected of having new-onset type 1 diabetes; comparing the concentrations of methylated biomarker DNA and unmethylated biomarker DNA in the sample with the concentrations of methylated biomarker DNA and unmethylated biomarker DNA in a control subject; and determining that the subject has new-onset type 1 diabetes when the concentrations of methylated biomarker DNA and unmethylated biomarker DNA in the sample is greater than the concentrations of methylated biomarker DNA and unmethylated biomarker DNA in the control subject.

In another aspect, the present disclosure is directed to a method for determining type 2 diabetes in a subject suspected of having type 2 diabetes. The method comprises: amplifying methylated preproinsulin (INS) DNA in a first sample obtained from the subject suspected of having type 2 diabetes; amplifying unmethylated preproinsulin (INS) DNA in the first sample obtained from the subject suspected of having type 2 diabetes; detecting whether a nucleotide located at position −69 from the preproinsulin (INS) transcriptional start site is methylated or unmethylated; amplifying methylated preproinsulin (INS) DNA in at least a second sample obtained from the subject suspected of having type 2 diabetes; amplifying unmethylated preproinsulin (INS) DNA in at least the second sample obtained from the subject suspected of having type 2 diabetes; detecting whether a nucleotide located at position −69 from the preproinsulin (INS) transcriptional start site is methylated or unmethylated; and determining that the subject has type 2 diabetes when the concentration of methylated preproinsulin (INS) DNA and unmethylated preproinsulin (INS) DNA in the first sample is greater than the concentration of methylated preproinsulin (INS) DNA and unmethylated preproinsulin (INS) DNA in the second sample.

In another aspect, the present disclosure is directed to a method for determining glucose tolerance impairment in a subject suspected of having glucose tolerance impairment. The method comprises: amplifying methylated preproinsulin (INS) DNA in a first sample obtained from the subject suspected of having glucose tolerance impairment; amplifying unmethylated preproinsulin (INS) DNA in the first sample obtained from the subject suspected of having glucose tolerance impairment; detecting whether a nucleotide located at position −69 from the preproinsulin (INS) transcriptional start site is methylated or unmethylated; amplifying methylated preproinsulin (INS) DNA in at least a second sample obtained from the subject suspected of having glucose tolerance impairment; amplifying unmethylated preproinsulin (INS) DNA in at least the second sample obtained from the subject suspected of having glucose tolerance impairment; detecting whether a nucleotide located at position −69 from the preproinsulin (INS) transcriptional start site is methylated or unmethylated; and determining that the subject has glucose tolerance impairment when the concentration of methylated preproinsulin (INS) DNA and unmethylated preproinsulin (INS) DNA in the first sample is greater than the concentration of methylated preproinsulin (INS) DNA and unmethylated preproinsulin (INS) DNA in the second sample.

In another aspect, the present disclosure is directed to a method for determining dysglycemia in an obese adolescent subject. The method includes amplifying preproinsulin (INS) DNA in a sample obtained from the obese adolescent subject; detecting whether a nucleotide located at position −69 from the preproinsulin (INS) transcriptional start site is methylated or unmethylated; comparing the concentration of methylated preproinsulin (INS) DNA in the sample obtained from the obese adolescent subject with the concentration of methylated preproinsulin (INS) DNA in an adolescent control subject selected from the group consisting of an adolescent control subject having normal weight, an adolescent control subject having normal glucose tolerance, and an adolescent having normal weight and normal glucose tolerance; and determining that the obese adolescent subject has dysglycemia when the concentration of methylated preproinsulin (INS) DNA is greater than the concentration of methylated preproinsulin (INS) DNA in the adolescent control subject.

In another aspect, the present disclosure is directed to a method for determining dysglycemia in an adolescent subject having type 2 diabetes. The method includes amplifying methylated preproinsulin (INS) DNA in a sample obtained from the obese adolescent subject; detecting whether a nucleotide located at position −69 from the preproinsulin (INS) transcriptional start site is methylated; comparing the concentration of methylated preproinsulin (INS) DNA in the sample obtained from the obese adolescent subject with the concentration of methylated preproinsulin (INS) DNA in an adolescent control subject selected from the group consisting of an adolescent control subject having normal weight, an adolescent control subject having normal glucose tolerance, and an adolescent having normal weight and normal glucose tolerance; and determining that the obese adolescent subject has dysglycemia when the concentration of methylated preproinsulin (INS) DNA is greater than the concentration of methylated preproinsulin (INS) DNA in the adolescent control subject.

In another aspect, the present disclosure is directed to a methylation-specific polymerase chain reaction assay for determining dysglycemia in an obese adolescent subject suspected of having dysglycemia. The method comprises: isolating DNA from a sample obtained from the obese adolescent subject suspected of having dysglycemia; treating the isolated DNA with bisulfite; amplifying methylated preproinsulin (INS) DNA in the sample; amplifying the preproinsulin (INS) promoter; and detecting whether a nucleotide located at position −69 from the preproinsulin (INS) transcriptional start site is methylated or unmethylated; comparing the concentration of methylated preproinsulin (INS) DNA in the sample obtained from the obese adolescent subject with the concentration of methylated preproinsulin (INS) DNA in an adolescent control subject selected from the group consisting of an adolescent control subject having normal weight, an adolescent control subject having normal glucose tolerance, and an adolescent having normal weight and normal glucose tolerance; and determining that the obese adolescent subject has dysglycemia when the concentration of methylated preproinsulin (INS) DNA in the sample is greater than the concentration of methylated preproinsulin (INS) DNA in the adolescent control subject.

In another aspect, the present disclosure is directed to a methylation-specific polymerase chain reaction assay for determining dysglycemia in an obese adolescent subject having type 2 diabetes and suspected of having dysglycemia. The method comprises: isolating DNA from a sample obtained from the obese adolescent subject having type 2 diabetes and suspected of having dysglycemia; treating the isolated DNA with bisulfite; amplifying methylated preproinsulin (INS) DNA in the sample; amplifying the preproinsulin (INS) promoter; and detecting whether a nucleotide located at position −69 from the preproinsulin (INS) transcriptional start site is methylated or unmethylated; comparing the concentration of methylated preproinsulin (INS) DNA in the sample obtained from the adolescent subject with the concentration of methylated preproinsulin (INS) DNA in an adolescent control subject selected from the group consisting of an adolescent control subject having normal weight, an adolescent control subject having normal glucose tolerance, and an adolescent having normal weight and normal glucose tolerance; and determining that the obese adolescent subject has dysglycemia when the concentration of methylated preproinsulin (INS) DNA is greater than the concentration of methylated preproinsulin (INS) DNA in the adolescent control subject.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts the methylation-specific PCR (MSP) assay methodology and validation used in the present disclosure. FIG. 1A depicts the MSP assay workflow for analysis of circulating unmethylated and methylated insulin DNA by ddPCR. FIGS. 1B & 1C depict dilutions of plasmids containing cloned, bisulfite-converted unmethylated and methylated INS DNA were subjected to ddPCR; 1-dimensional plots from ddPCR are shown for fluorescent probes specific for unmethylated INS DNA (FIG. 1B) and methylated INS DNA (FIG. 1C). In FIGS. 1B & 1C, the positive, negative, and cross-reactive populations are identified. FIG. 1D depicts the quantitation of plasmid dilution curves, presented as copies/μl; r²=0.9818 for unmethylated INS DNA; r²=0.9685 for methylated INS DNA. FIG. 1E depicts 2-dimensional plots using plasmid standards for unmethylated and methylated INS DNA, and for a 1:1 mixture of the two plasmids. Arrows identify the unmethylated, methylated, and unmethylated+methylated (double-positive) INS DNA-containing droplets.

FIG. 2 depicts circulating human unmethylated and methylated INS DNA levels following human islet transplantation in immunocompetent mice. FIGS. 2A & 2B refer to CD1 mice (n=4) that were either untransplanted (No Transplant) or transplanted with 200 human islets beneath the kidney capsule (Transplant). Serum was collected at the time points indicated and processed for MSP assay for either human INS or mouse INS2 DNA using ddPCR. FIG. 2A depicts circulating unmethylated DNA levels. FIG. 2B depicts circulating methylated DNA levels. *p<0.05 compared to time 0. FIGS. 2C & 2D refer to serum from NOD, NOD-SCID, and CD1 mice (n=3 per group) that were collected at the ages indicated and at the age NOD mice developed diabetes (12-14 weeks), and processed for MSP assay. FIG. 2C depicts circulating unmethylated INS2 DNA levels measured by ddPCR. FIG. 2D depicts circulating methylated INS2 DNA levels measured by ddPCR. *p<0.05 compared to time 0 in panels (A) and (B), and * p<0.05 compared to CD1 mice at the corresponding age in panels (C) and (D).

FIG. 3 depicts representative 2D and1D ddPCR plots from control and T1D subjects. FIG. 3A depicts a 2D plot from a representative control subject. FIG. 3B depicts a 2D plot from a representative new-onset T1D subject. Arrows identify the respective unmethlylated, methylated, and unmethylated+methylated (double-positive) INS-containing droplets. FIG. 3C depicts representative 1D plots for methylated INS DNA from two control and two new-onset T1D subjects. FIG. 3D depicts representative 1D plots for unmethylated INS DNA from two control and two new-onset T1D subjects. In FIGS. 3C & 3D, the positive, negative, and overlap (FAM probe overlapping into the VIC channel, and vice versa) signals are identified.

FIG. 4 depicts circulating unmethylated and methylated INS DNA levels in human cohorts. FIG. 4A depicts circulating unmethylated INS DNA levels in human cohorts depicted as log(copies/μ1). FIG. 4B depicts longitudinal change in circulating unmethylated INS DNA levels in pediatric T1D subjects at diagnosis, 8 weeks following diagnosis, and 1 year following diagnosis. FIG. 4C depicts circulating methylated INS DNA levels in human cohorts depicted as log(copies/μ1). FIG. 4D depicts longitudinal change in circulating methylated INS DNA levels in pediatric T1D subjects at diagnosis, 8 weeks following diagnosis, and 1 year following diagnosis. *p<0.05, *** p<0.0001, ** p<0.001, ns=not significant (p>0.05).

FIG. 5A depicts data from the analysis of the Infinium HumanMethylation 450 Array of 64 human islet preparations vs. 27 human tissue controls. Shown are the 10 genes that exhibited the greatest differential methylation from over 6000 differentially methylated genes.

FIG. 5B depicts the 10 hypo- and hyper-methylated genes that did not have any overlap in any sample between islets and control tissues.

FIG. 6 depicts the elevation in methylated and unmethylated INS DNA levels in the urine of new-onset T1D. 2-dimensional plots from ddPCR are shown for a control and new-onset T1D subject from whom analysis was performed from urine for unmethylated and methylated INS DNA. Circled populations identify the unmethylated, methylated, and unmethylated+methylated double-positive) INS DNA-containing droplets. Note that droplets from the T1D subject are greater for both unmethylated and methylated (and double positive) compared to the control subject.

FIG. 7A depicts unmethylated INS DNA in cell-free DNA isolated from serum was observed in subjects with T2D, subjects with impaired glucose tolerance (IGT) and subjects with normal glucose tolerance (NGT).

FIG. 7B depicts methylated INS DNA in cell-free DNA isolated from serum was observed in subjects with T2D, subjects with impaired glucose tolerance (IGT) and subjects with normal glucose tolerance (NGT).

FIGS. 8A & 8B depict the ability of the mouse MSP assay to distinguish unmethylated Ins2 using the FAM-labeled probe and methylated Ins2 using the VIC-labeled probe in a linear fashion.

FIG. 8C depicts the detection of unmethylated Ins2 in DNA-spiked serum.

FIG. 8D depicts the detection of methylated Ins2 in DNA-spiked serum.

FIG. 9A depicts increased body weight values in HFD-fed BL6 mice by 6 weeks post start of diet.

FIG. 9B depicts increased fasting blood glucose values in HFD-fed BL6 mice by 6 weeks post start of diet.

FIG. 9C depicts impaired glucose tolerance by GTT in HFD-fed BL6 mice as early as 2 weeks post start of diet.

FIG. 9D depicts increased β cell mass in HFD-fed animals by 6 weeks of age compared to LFD-fed animals.

FIG. 9E depicts the increase in unmethylated Ins2 DNA after STZ injections.

FIG. 9F depicts the increase in methylated Ins2 DNA after STZ injections.

FIGS. 10A and 10B are graphs depicting values for both unmethylated and methylated INS increased with age (P<0.001, R=0.229; P<0.001, R=0.3201).

FIGS. 11A and 11B are graphs depicting values for both unmethylated and methylated INS in normal glucose tolerance (NGT) adults, impaired glucose tolerance (IGT) adults and adults with Type 2 diabetes (T2D).

FIGS. 12A and 12B are graphs depicting values for both unmethylated and methylated INS for triplicate experiments in normal glucose tolerance (NGT) adults, impaired glucose tolerance (IGT) adults and adults with Type 2 diabetes (T2D).

FIGS. 13A and 13B are graphs depicting values for both unmethylated and methylated INS in normal weight-normal glucose tolerance (NW-NGT) youth, obese adolescents with NGT (OB-NGT), obese adolescents with IGT (OB-IGT), obese adolescents with T2D without islet autoantibodies (Ab-T2DM) and obese adolescent subjects with islet autoantibodies (Ab+T2DM) (P<0.001; P<0.01).

FIGS. 14A and 14B are graphs depicting values for both unmethylated and methylated INS for triplicate experiments in lean NGT youth, obese adolescents with NGT, obese adolescents with IGT, obese adolescents with T2D without islet autoantibodies (AAb-T2D) and obese adolescent subjects with islet autoantibodies (AAb+T2D).

FIGS. 15A and 15B are graphs depicting unmethylated and methylated INS DNA correlated with HbA1c in adolescent subjects.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

As used herein, “new-onset type 1 diabetes” refers to a diagnosis of type 1 diabetes within two days of diagnosis. Specific diagnostic criteria by the American Diabetes Association include: a fasting plasma glucose level ≥126 mg/dL (7.0 mmol/L), or a 2-hour plasma glucose level ≥200 mg/dL (11.1 mmol/L) during a 75-g oral glucose tolerance test, or a random plasma glucose ≥200 mg/dL (11.1 mmol/L) in a subject with classic symptoms of hyperglycemia or hyperglycemic crisis. “Recent onset type 1 diabetes” as understood by those skilled in the art refers to type 1 diabetes subjects within 4-18 months of diagnosis.

As used herein, “a subject in need thereof” (also used interchangeably herein with “a patient in need thereof”) refers to a subject susceptible to or at risk of a specified disease, disorder, or condition. The methods of screening circulating methylated and unmethylated DNA can be used with a subset of subjects who are susceptible to or at elevated risk for experiencing diseases resulting from abnormalities in blood glucose levels, but are also relevant to different stages of disease progression. In the present disclosure, the methods of screening methylated preproinsulin (INS) DNA and unmethylated preproinsulin (INS) DNA can be used with a subset of subjects who are susceptible to or at elevated risk for experiencing dysglycemia, type 1 diabetes, impaired glucose tolerance (IGT, interchangeably used herein with glucose tolerance impairment), type 1 diabetes (T1D), new-onset type 1 diabetes, type 2 diabetes (T2D), and obesity. Subjects may be susceptible to or at elevated risk for dysglycemia, type 1 diabetes, impaired glucose tolerance, T1D, new-onset T1D, T2D, and obesity due to family history, age, environment, clinical presentation, polyuria, polydipsia, polyphagia, unintentional weight loss, diabetic ketoacidosis, personal or family history of autoimmune disorders, lifestyle hyperglycemia, and/or obesity. In some embodiments, particularly suitable subjects in need are obese adolescent subjects. In some embodiments, more particularly suitable obese adolescent subjects are obese adolescent subjects with normal glucose tolerance, obese adolescent subjects with impaired glucose tolerance, obese adolescent subjects with T2D, obese adolescent subjects with islet autoantibodies, and obese adolescent subjects with T2D and islet autoantibodies.

Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

As used herein, “susceptible” and “at risk” refer to having little resistance to a certain disease, disorder or condition, including being genetically predisposed, having a family history of, and/or having symptoms of the disease, disorder or condition.

Oligonucleotide Probes

In one aspect, the present disclosure is directed to oligonucleotide probes. In one embodiment, the oligonucleotide probe includes SEQ ID NO:1 and a label. In another embodiment, the oligonucleotide probe includes SEQ ID NO:2 and a label. In one embodiment, the oligonucleotide probe includes SEQ ID NO:3 and a label. In one embodiment, the oligonucleotide probe includes SEQ ID NO:4 and a label. In another embodiment, the present disclosure is directed to a primer pair. The primer pair includes a first oligonucleotide probe including SEQ ID NO:3 and a label and a second oligonucleotide probe including SEQ ID NO:4 and a label. In another embodiment, the first oligonucleotide probe and the second oligonucleotide probe can independently include second labels.

Suitable labels can be reporter dyes, fluorescent dyes, nonfluorescent quenchers, and combinations thereof.

Suitable reporter dyes can be, for example, 6-carboxyfluorescein, 6-carboxy-X-rhodamine, tetrachlorofluorescein, hexachloro-fluorescein, and 6-carboxy-4′,5′-dichloro-2′,7′-dimethoxyfluorescein. Particularly suitable reporter dyes can be VIC®, FAM®, TET™, JOE™, ABY®, JUN®, NED™, ROX, CY3®, CY5®, ABY, and combinations thereof.

Suitable nonfluorescent quenchers can be, for example, tetramethylrhodamine. Nonfluorescent quenchers can further include a minor groove binding ligand (MGB). The nonfluorescent molecule quenches the fluorescence emitted by the fluorophore when excited by the cycler's light source. Nonfluorescent quenchers can provide lower background signal and result in better precision in quantitation. The MGB moiety stabilizes the hybridized probe and raises the melting temperature. Nonfluorescent quenchers can be, for example, TAMRA™, QSY®, NFQ-MGB and combinations thereof.

In another aspect, the present disclosure is directed to use of an oligonucleotide of SEQ ID NO:3 in the determination of a methylated preproinsulin DNA in a sample. In another aspect, the present disclosure is directed to use of an oligonucleotide of SEQ ID NO:4 in the determination of an unmethylated preproinsulin DNA in a sample.

In another aspect, the present disclosure is directed to a diagnostic kit including one or more of the group consisting of an oligonucleotide of SEQ ID NO:1, an oligonucleotide of SEQ ID NO:2, an oligonucleotide of SEQ ID NO:3, and an oligonucleotide of SEQ ID NO:4.

In another aspect, the present disclosure is directed to an assay device including one or more of the group consisting of an oligonucleotide of SEQ ID NO:1, an oligonucleotide of SEQ ID NO:2, an oligonucleotide of SEQ ID NO:3, and an oligonucleotide of SEQ ID NO:4.

Use of a Circulating Unmethylated DNA and Methylated DNA

In another aspect, the present disclosure is directed to use of a circulating unmethylated DNA, a circulating methylated DNA, and combinations thereof as a biomarker for one or more of the group consisting of Type 1 diabetes, new-onset Type 1 diabetes, Type 2 diabetes, impaired glucose tolerance, and dysglycemia.

Suitable methylated and unmethylated DNA includes methylated and unmethylated preproinsulin (INS) DNA; methylated and unmethylated chr3: 125085322 (ZNF148, zinc finger protein 148) DNA; methylated and unmethylated chr3:12586368 (intergenic) DNA; methylated and unmethylated ch1:153610672 (Clorf77, chromosome 1, open reading frame 77) DNA; methylated and unmethylated chr3: 135702110 (PPP2R3A; Serine/threonine-protein phosphatase 2A regulatory subunit B) DNA; methylated and unmethylated chr2:189064557 (intergenic) DNA; methylated and unmethylated chr14:105491309 (intergenic) DNA; methylated and unmethylated chr5:35046992 (AGXT2, alanine-glyoxylate aminotransferase 2) DNA; methylated and unmethylated chr7:107300287 (SLC26A4, Solute Carrier Family 26 (Anion Exchanger), Member 4) DNA; methylated and unmethylated chr8:126649807 (intergenic) DNA; methylated and unmethylated chr12:49759545 (SPATS2, spermatogenesis associated, serine rich 2) DNA; and combinations thereof.

Methods for Diagnosing New-Onset Type 1 Diabetes in a Subject

In another aspect, the present disclosure is directed to a method for diagnosing new-onset type 1 diabetes in a subject suspected of having new-onset type 1 diabetes. The method includes amplifying methylated preproinsulin (INS) DNA in a sample obtained from the subject suspected of having new-onset type 1 diabetes; amplifying unmethylated preproinsulin (INS) DNA in the sample obtained from the subject suspected of having new-onset type 1 diabetes; detecting whether a nucleotide located at position −69 from the preproinsulin (INS) transcriptional start site is methylated or unmethylated; comparing the concentration of methylated preproinsulin (INS) DNA and unmethylated preproinsulin (INS) DNA in the sample with the concentration of methylated preproinsulin (INS) DNA and unmethylated preproinsulin (INS) DNA in a control subject; and determining that the subject has new-onset type 1 diabetes when the concentration of methylated preproinsulin (INS) DNA and unmethylated preproinsulin (INS) DNA in the sample is greater than the concentration of methylated preproinsulin (INS) DNA and unmethylated preproinsulin (INS) DNA in the control subject.

A particularly suitable reference sequence for identifying position −69 can be found in the preproinsulin (INS) gene having the GenBank Accession number V00565 (GI:33930; Ensembl number: ENSG00000254647; provided herein as SEQ ID NO:9).

Suitable amplification methods are known to those skilled in the art such as, for example, polymerase chain reaction and isothermal amplification methods. Suitable polymerase chain reaction methods for amplifying preproinsulin (INS) DNA are known to those skilled in the art. A particularly suitable amplification method is Droplet Digital™ PCR (ddPCR™). ddPCR™ technology employs the analysis of discrete individual PCR reactions (up to 20,000/sample) to identify the absence or presence of the target DNA, and subsequently utilizes Poisson statistics to extrapolate the number of copies of the target DNA in the sample.

In one aspect, the nucleotide located at position −69 from the preproinsulin (INS) transcriptional start site is cytosine.

In one aspect, the method includes amplifying the methylated preproinsulin (INS) DNA in the sample using an oligonucleotide comprising SEQ ID NO:3. In another aspect, the method includes amplifying the unmethylated preproinsulin (INS) DNA in the sample using an oligonucleotide comprising SEQ ID NO:4. In another aspect, the methylated preproinsulin (INS) DNA in the sample the unmethylated preproinsulin (INS) DNA in the sample is amplified using a primer pair, wherein the primer pair includes an oligonucleotide comprising SEQ ID NO:3 and an oligonucleotide comprising SEQ ID NO:4.

In another aspect, the method further includes amplifying the preproinsulin (INS) promoter. The preproinsulin (INS) promoter can be amplified using a first oligonucleotide comprising SEQ ID NO:1 and a second oligonucleotide comprising SEQ ID NO:2.

In another aspect, the method further includes determining the concentration of methylated preproinsulin (INS) DNA. The methylated preproinsulin (INS) DNA is methylated at a nucleotide located at position −69 from the preproinsulin (INS) transcriptional start site. The nucleotide located at position −69 from the preproinsulin (INS) transcriptional start site is cytosine. As described herein, statistics such as, for example, Poisson statistics, can be used to extrapolate the number of copies, and thus, the concentration of the methylated and unmethylated preproinsulin (INS) DNA in the sample.

In another aspect, the method further includes determining the concentration of unmethylated preproinsulin (INS) DNA. The unmethylated preproinsulin (INS) DNA is methylated at a nucleotide located at position −69 from the preproinsulin (INS) transcriptional start site. The nucleotide located at position −69 from the preproinsulin (INS) transcriptional start site is cytosine.

In another aspect, the method includes subjecting (i.e., treating) the preproinsulin (INS) DNA in the sample to a bisulfite reaction. The preproinsulin (INS) DNA can suitably be treated alone after isolation and purification from the sample and the preproinsulin (INS) DNA can suitably be treated with all (the total) or part of the DNA in the sample. The preproinsulin (INS) DNA is subjected to a bisulfite reaction by treating the preproinsulin (INS) DNA and/or the total DNA with bisulfite. The bisulfite treatment can be performed using standard methods such as, for example, EZ DNA METHYLATION™ kit (commercially available from Zymo Research, Irvine, Calif.) and EZ DNA METHYLATION-LIGHTNING Kit (commercially available from Zymo Research, Irvine, Calif.). Treatment of DNA with bisulfite results in the conversion of unmethylated cytosines to uracils.

In another aspect, the copy number per microliter of methylated preproinsulin (INS) DNA and the copy number per microliter of unmethylated preproinsulin (INS) DNA are determined.

Suitable samples can be serum, plasma, whole blood and urine. Particularly suitable samples include serum, plasma and urine. Total DNA and preproinsulin (INS) DNA can be extracted from serum and plasma using standard methods such as, for example, ZR SERUM DNA Kit™ (commercially available from Zymo Research, Irvine, Calif.) and QIAamp DNA blood mini kit (commercially available from QIAGEN, Germantown, Md.). Preproinsulin (INS) DNA can be extracted from urine using standard methods such as, for example, ZR URINE DNA Kit™ (commercially available from Zymo Research, Irvine, Calif.), for example.

The concentration of methylated DNA and unmethylated DNA can be determined by measuring fluorescence. The concentration of methylated preproinsulin (INS) DNA and unmethylated preproinsulin (INS) DNA can be determined by measuring fluorescence. Fluorescence can be measured at 518 nm, 548 nm, and 582.

Suitable control subjects include, for example, a healthy pediatric subject, a subject having type 1 diabetes for at least 8 weeks, a subject having type 1 diabetes for at least one year, a healthy adult subject, an adult with obesity, an adult having type 2 diabetes, an adult having auto-immune hepatitis, and combinations thereof.

In another embodiment, the method further includes comparing the concentrations of methylated and unmethylated preproinsulin (INS) DNA; methylated and unmethylated chr3: 125085322 (ZNF148, zinc finger protein 148) DNA; methylated and unmethylated chr3:12586368 (intergenic) DNA; methylated and unmethylated ch1:153610672 (Clorf77, chromosome 1, open reading frame 77) DNA; methylated and unmethylated chr3: 135702110 (PPP2R3A; Serine/threonine-protein phosphatase 2A regulatory subunit B) DNA; methylated and unmethylated chr2:189064557 (intergenic) DNA; methylated and unmethylated chr14:105491309 (intergenic) DNA; methylated and unmethylated chr5:35046992 (AGXT2, alanine-glyoxylate aminotransferase 2) DNA; methylated and unmethylated chr7:107300287 (SLC26A4, Solute Carrier Family 26 (Anion Exchanger), Member 4) DNA; methylated and unmethylated chr8:126649807 (intergenic) DNA; methylated and unmethylated chr12:49759545 (SPATS2, spermatogenesis associated, serine rich 2) DNA; and combinations thereof.

A Methylation-Specific Polymerase Chain Reaction Assay for Determining New-Onset Type 1 Diabetes

In another aspect, the present disclosure is directed to a methylation-specific polymerase chain reaction assay for determining new-onset type 1 diabetes in a subject suspected of having new-onset type 1 diabetes. The method includes isolating DNA from a sample obtained from a subject suspected of having new-onset type 1 diabetes; treating the isolated DNA with bisulfite; amplifying unmethylated preproinsulin (INS) DNA in the sample; amplifying methylated preproinsulin (INS) DNA in the sample; amplifying the preproinsulin (INS) promoter; and detecting whether a nucleotide located at position −69 from the preproinsulin (INS) transcriptional start site is methylated or unmethylated.

In one aspect, the method includes amplifying the methylated preproinsulin (INS) DNA in the sample using an oligonucleotide comprising SEQ ID NO:3. In another aspect, the method includes amplifying the unmethylated preproinsulin (INS) DNA in the sample using an oligonucleotide comprising SEQ ID NO:4. In another aspect, the methylated preproinsulin (INS) DNA in the sample the unmethylated preproinsulin (INS) DNA in the sample is amplified using a primer pair, wherein the primer pair includes an oligonucleotide comprising SEQ ID NO:3 and an oligonucleotide comprising SEQ ID NO:4.

The preproinsulin (INS) promoter can be amplified using a first oligonucleotide comprising SEQ ID NO:1 and a second oligonucleotide comprising SEQ ID NO:2.

In another aspect, the methylated preproinsulin (INS) DNA in the sample the unmethylated preproinsulin (INS) DNA in the sample is amplified using a primer pair, wherein the primer pair includes an oligonucleotide comprising SEQ ID NO:3 and an oligonucleotide comprising SEQ ID NO:4; and the preproinsulin (INS) promoter in the sample is amplified using a primer pair, wherein the primer pair includes a first oligonucleotide comprising SEQ ID NO:1 and a second oligonucleotide comprising SEQ ID NO:2.

In one aspect, the nucleotide located at position −69 from the preproinsulin (INS) transcriptional start site is cytosine.

In another aspect, the method further includes determining the concentration of methylated preproinsulin (INS) DNA. The methylated preproinsulin (INS) DNA is methylated at a nucleotide located at position −69 from the preproinsulin (INS) transcriptional start site. The nucleotide located at position −69 from the preproinsulin (INS) transcriptional start site is cytosine.

In another aspect, the method further includes determining the concentration of unmethylated preproinsulin (INS) DNA. The unmethylated preproinsulin (INS) DNA is methylated at a nucleotide located at position −69 from the preproinsulin (INS) transcriptional start site. The nucleotide located at position −69 from the preproinsulin (INS) transcriptional start site is cytosine.

In another aspect, the method includes subjecting (i.e., treating) the preproinsulin (INS) DNA in the sample to a bisulfite reaction as described herein. The preproinsulin (INS) DNA is subjected to a bisulfite reaction by treating the preproinsulin (INS) DNA with bisulfite using standard methods as described herein.

Suitable samples can be serum, plasma, whole blood and urine. Particularly suitable samples include serum, plasma and urine. Preproinsulin (INS) DNA can be extracted from serum and plasma using standard methods as described herein. Preproinsulin (INS) DNA can be extracted from urine using standard methods as described herein.

Particularly suitable amplification of preproinsulin (INS) DNA uses Droplet Digital™ PCR (ddPCR™) as described herein.

In a particularly suitable embodiment, the PCR is performed using the following cycling conditions: 95° C. for 10 minutes, 94° C. for 30 seconds, and 57.5° C. for 60 seconds for 40 amplification cycles.

In another aspect, the method further includes comparing the concentration of methylated preproinsulin (INS) DNA and the concentration of unmethylated preproinsulin (INS) DNA from the subject to the concentration of methylated preproinsulin (INS) DNA to the concentration of methylated preproinsulin (INS) DNA and the concentration of unmethylated preproinsulin (INS) DNA obtained from a control subject; and determining the subject as being suspected of having type 1 diabetes if the concentration of methylated preproinsulin (INS) DNA and the concentration of unmethylated preproinsulin (INS) DNA from the subject is elevated when compared to the concentration of methylated preproinsulin (INS) DNA and the concentration of unmethylated preproinsulin (INS) DNA of the control subject.

Suitable control subjects include, for example, a healthy pediatric subject, a subject having type 1 diabetes for at least 8 weeks, a subject having type 1 diabetes for at least one year, a healthy adult subject, an adult with obesity, an adult having type 2 diabetes, an adult having auto-immune hepatitis, and combinations thereof.

In another embodiment, the method further includes comparing the concentrations of methylated and unmethylated preproinsulin (INS) DNA; methylated and unmethylated chr3: 125085322 (ZNF148, zinc finger protein 148) DNA; methylated and unmethylated chr3:12586368 (intergenic) DNA; methylated and unmethylated ch1:153610672 (Clorf77, chromosome 1, open reading frame 77) DNA; methylated and unmethylated chr3: 135702110 (PPP2R3A; Serine/threonine-protein phosphatase 2A regulatory subunit B) DNA; methylated and unmethylated chr2:189064557 (intergenic) DNA; methylated and unmethylated chr14:105491309 (intergenic) DNA; methylated and unmethylated chr5:35046992 (AGXT2, alanine-glyoxylate aminotransferase 2) DNA; methylated and unmethylated chr7:107300287 (SLC26A4, Solute Carrier Family 26 (Anion Exchanger), Member 4) DNA; methylated and unmethylated chr8:126649807 (intergenic) DNA; methylated and unmethylated chr12:49759545 (SPATS2, spermatogenesis associated, serine rich 2) DNA; and combinations thereof.

Methods for Distinguishing Between Type 1 Diabetes and Type 2 Diabetes in a Subject Suspected of Having Type 1 Diabetes

In another aspect, the present disclosure is directed to a method for distinguishing between type 1 diabetes and type 2 diabetes in a subject suspected of having type 1 diabetes. The method includes amplifying methylated preproinsulin (INS) DNA in a sample obtained from the subject suspected of having type 1 diabetes; amplifying unmethylated preproinsulin (INS) DNA in the sample obtained from the subject suspected of having new-onset type 1 diabetes; detecting whether a nucleotide located at position −69 from the preproinsulin (INS) transcriptional start site is methylated or unmethylated; comparing the concentration of methylated preproinsulin (INS) DNA and the concentration of unmethylated preproinsulin (INS) DNA from the subject to the concentration of methylated preproinsulin (INS) DNA and the concentration of unmethylated preproinsulin (INS) DNA from a subject having type 2 diabetes; and diagnosing the subject as being suspected of having type 1 diabetes if the concentration of methylated preproinsulin (INS) DNA and the concentration of unmethylated preproinsulin (INS) DNA from the subject are elevated when compared to the concentration of methylated preproinsulin (INS) DNA and the concentration of unmethylated preproinsulin (INS) DNA of the subject having type 2 diabetes.

In one aspect, the nucleotide located at position −69 from the preproinsulin (INS) transcriptional start site is cytosine.

In one aspect, the method includes amplifying the methylated preproinsulin (INS) DNA in the sample using an oligonucleotide comprising SEQ ID NO:3. In another aspect, the method includes amplifying the unmethylated preproinsulin (INS) DNA in the sample using an oligonucleotide comprising SEQ ID NO:4. In another aspect, the methylated preproinsulin (INS) DNA in the sample the unmethylated preproinsulin (INS) DNA in the sample is amplified using a primer pair, wherein the primer pair includes an oligonucleotide comprising SEQ ID NO:3 and an oligonucleotide comprising SEQ ID NO:4.

In another aspect, the method further includes amplifying the preproinsulin (INS) promoter. The preproinsulin (INS) promoter can be amplified using a first oligonucleotide comprising SEQ ID NO:1 and a second oligonucleotide comprising SEQ ID NO:2.

In another aspect, the method further includes determining the concentration of methylated preproinsulin (INS) DNA. The methylated preproinsulin (INS) DNA is methylated at a nucleotide located at position −69 from the preproinsulin (INS) transcriptional start site. The nucleotide located at position −69 from the preproinsulin (INS) transcriptional start site is cytosine.

In another aspect, the method further includes determining the concentration of unmethylated preproinsulin (INS) DNA. The unmethylated preproinsulin (INS) DNA is methylated at a nucleotide located at position −69 from the preproinsulin (INS) transcriptional start site. The nucleotide located at position −69 from the preproinsulin (INS) transcriptional start site is cytosine.

In another aspect, the method includes subjecting (i.e., treating) the preproinsulin (INS) DNA in the sample to a bisulfite reaction. The preproinsulin (INS) DNA is subjected to a bisulfite reaction by treating the preproinsulin (INS) DNA with bisulfite using standard methods as described herein.

Suitable samples can be serum, plasma, whole blood and urine. Particularly suitable samples include serum, plasma and urine. Preproinsulin (INS) DNA can be extracted from serum and plasma using standard methods as described herein.

The concentration of methylated DNA and unmethylated DNA can be determined by measuring fluorescence. The concentration of methylated preproinsulin (INS) DNA and unmethylated preproinsulin (INS) DNA can be determined by measuring fluorescence. Fluorescence can be measured at 518 nm, 548 nm, and 582.

In another aspect, the method further includes comparing the concentration of methylated preproinsulin (INS) DNA and the concentration of unmethylated preproinsulin (INS) DNA from the subject to the concentration of methylated preproinsulin (INS) DNA to the concentration of methylated preproinsulin (INS) DNA and the concentration of unmethylated preproinsulin (INS) DNA obtained from a control subject; and diagnosing the subject as being suspected of having type 1 diabetes if the concentration of methylated preproinsulin (INS) DNA and the concentration of unmethylated preproinsulin (INS) DNA from the subject is elevated when compared to the concentration of methylated preproinsulin (INS) DNA and the concentration of unmethylated preproinsulin (INS) DNA of the control subject.

Suitable control subjects include, for example, a healthy pediatric subject, a subject having type 1 diabetes for at least 8 weeks, a subject having type 1 diabetes for at least one year, a healthy adult subject, an adult with obesity, an adult having type 2 diabetes, an adult having auto-immune hepatitis, and combinations thereof.

In another embodiment, the method further includes comparing the concentrations of methylated and unmethylated preproinsulin (INS) DNA; methylated and unmethylated chr3: 125085322 (ZNF148, zinc finger protein 148) DNA; methylated and unmethylated chr3:12586368 (intergenic) DNA; methylated and unmethylated ch1:153610672 (Clorf77, chromosome 1, open reading frame 77) DNA; methylated and unmethylated chr3: 135702110 (PPP2R3A; Serine/threonine-protein phosphatase 2A regulatory subunit B) DNA; methylated and unmethylated chr2:189064557 (intergenic) DNA; methylated and unmethylated chr14:105491309 (intergenic) DNA; methylated and unmethylated chr5:35046992 (AGXT2, alanine-glyoxylate aminotransferase 2) DNA; methylated and unmethylated chr7:107300287 (SLC26A4, Solute Carrier Family 26 (Anion Exchanger), Member 4) DNA; methylated and unmethylated chr8:126649807 (intergenic) DNA; methylated and unmethylated chr12:49759545 (SPATS2, spermatogenesis associated, serine rich 2) DNA; and combinations thereof.

Methods for Determining Type 2 Diabetes in a Subject Suspected of Having Type 2 Diabetes

In another aspect, the present disclosure is directed to a method for determining type 2 diabetes in a subject suspected of having type 2 diabetes. The method includes: amplifying methylated preproinsulin (INS) DNA in a first sample obtained from the subject suspected of having type 2 diabetes; amplifying unmethylated preproinsulin (INS) DNA in the first sample obtained from the subject suspected of having type 2 diabetes; detecting whether a nucleotide located at position −69 from the preproinsulin (INS) transcriptional start site is methylated or unmethylated; amplifying methylated preproinsulin (INS) DNA in at least a second sample obtained from the subject suspected of having type 2 diabetes; amplifying unmethylated preproinsulin (INS) DNA in at least the second sample obtained from the subject suspected of having type 2 diabetes; detecting whether a nucleotide located at position −69 from the preproinsulin (INS) transcriptional start site is methylated or unmethylated; and determining that the subject has type 2 diabetes when the concentration of methylated preproinsulin (INS) DNA and unmethylated preproinsulin (INS) DNA in the first sample is greater than the concentration of methylated preproinsulin (INS) DNA and unmethylated preproinsulin (INS) DNA in the second sample.

In another aspect, the method includes subjecting (i.e., treating) the preproinsulin (INS) DNA in the sample to a bisulfite reaction. The preproinsulin (INS) DNA is subjected to a bisulfite reaction by treating the preproinsulin (INS) DNA with bisulfite using standard methods as described herein.

Suitable samples can be serum, plasma, whole blood and urine. Particularly suitable samples include serum, plasma and urine. Preproinsulin (INS) DNA can be extracted from serum and plasma using standard methods as described herein.

Method for Determining Glucose Tolerance Impairment in a Subject Suspected of Having Glucose Tolerance Impairment

In another aspect, the present disclosure is directed to a method for determining glucose tolerance impairment in a subject suspected of having glucose tolerance impairment. The method includes: amplifying methylated preproinsulin (INS) DNA in a first sample obtained from the subject suspected of having glucose tolerance impairment; amplifying unmethylated preproinsulin (INS) DNA in a first sample obtained from the subject suspected of having glucose tolerance impairment; detecting whether a nucleotide located at position −69 from the preproinsulin (INS) transcriptional start site is methylated or unmethylated; amplifying methylated preproinsulin (INS) DNA in at least a second sample obtained from the subject suspected of having glucose tolerance impairment; amplifying unmethylated preproinsulin (INS) DNA in at least a second sample obtained from the subject suspected of having glucose tolerance impairment; detecting whether a nucleotide located at position −69 from the preproinsulin (INS) transcriptional start site is methylated or unmethylated; and determining that the subject has glucose tolerance impairment when the concentration of methylated preproinsulin (INS) DNA and unmethylated preproinsulin (INS) DNA in the first sample is greater than the concentration of methylated preproinsulin (INS) DNA and unmethylated preproinsulin (INS) DNA in the second sample.

In another aspect, the method includes subjecting (i.e., treating) the preproinsulin (INS) DNA in the sample to a bisulfite reaction. The preproinsulin (INS) DNA is subjected to a bisulfite reaction by treating the preproinsulin (INS) DNA with bisulfite using standard methods as described herein.

Suitable samples can be serum, plasma, whole blood and urine. Particularly suitable samples include serum, plasma and urine. Preproinsulin (INS) DNA can be extracted from serum and plasma using standard methods as described herein.

Methods for Determining Dysglycemia in an Obese Adolescent Subject

In another aspect, the present disclosure is directed to a method for determining dysglycemia in an obese adolescent subject. The method includes amplifying preproinsulin (INS) DNA in a sample obtained from the obese adolescent subject; detecting whether a nucleotide located at position −69 from the preproinsulin (INS) transcriptional start site is methylated or unmethylated; comparing the concentration of methylated preproinsulin (INS) DNA in the sample obtained from the obese adolescent subject with the concentration of methylated preproinsulin (INS) DNA in an adolescent control subject selected from the group consisting of an adolescent control subject having normal weight, an adolescent control subject having normal glucose tolerance, and an adolescent having normal weight and normal glucose tolerance; and determining that the obese adolescent subject has dysglycemia when the concentration of methylated preproinsulin (INS) DNA is greater than the concentration of methylated preproinsulin (INS) DNA in the adolescent control subject.

In one embodiment, the obese adolescent subject has normal glucose tolerance. In another embodiment, the obese adolescent subject has impaired glucose tolerance.

As used herein, “adolescent” and “adolescence” refers to a youth in the period of growth and development between about puberty and adulthood. As recognized by the World Health Organization, adolescence is the period in human growth and development that occurs after childhood and before adulthood, from the age of about 10 years of age to about 19 years of age. As used herein, “obese” refers to having excess body fat as recognized by the Centers for Disease Control and Prevention. Guidance for estimating obesity can use body mass index (BMI) calculation from the weight and height of the subject. Obesity as determined by BMI of a subject is estimated as having a BMI greater than the 95^{th} percentile for age and gender.

Methods for Determining Dysglycemia in an Adolescent Subject Having Type 2 Diabetes

In another aspect, the present disclosure is directed to a method for determining dysglycemia in an obese adolescent subject having type 2 diabetes. The method includes amplifying methylated preproinsulin (INS) DNA in a sample obtained from the obese adolescent subject; detecting whether a nucleotide located at position −69 from the preproinsulin (INS) transcriptional start site is methylated; comparing the concentration of methylated preproinsulin (INS) DNA in the sample obtained from the obese adolescent subject with the concentration of methylated preproinsulin (INS) DNA in an adolescent control subject selected from the group consisting of an adolescent control subject having normal weight, an adolescent control subject having normal glucose tolerance, and an adolescent having normal weight and normal glucose tolerance; and determining that the obese adolescent subject has dysglycemia when the concentration of methylated preproinsulin (INS) DNA is greater than the concentration of methylated preproinsulin (INS) DNA in the adolescent control subject.

In another embodiment, the obese adolescent subject has type 2 diabetes and islet autoantibodies.

In another embodiment, the method further includes amplifying unmethylated preproinsulin (INS) DNA in a sample obtained from the obese adolescent subject; detecting whether a nucleotide located at position −69 from the preproinsulin (INS) transcriptional start site is unmethylated; comparing the concentration of unmethylated preproinsulin (INS) DNA in the sample obtained from the obese adolescent subject with the concentration of unmethylated preproinsulin (INS) DNA in an adolescent control subject selected from the group consisting of an adolescent control subject having normal weight, an adolescent control subject having normal glucose tolerance, and an adolescent having normal weight and normal glucose tolerance; and determining that the obese adolescent subject has dysglycemia when the concentration of unmethylated preproinsulin (INS) DNA is greater than the concentration of unmethylated preproinsulin (INS) DNA in the adolescent control subject.

A Methylation-Specific Polymerase Chain Reaction Assay for Determining Dysglycemia in an Obese Adolescent Subject Suspected of Having Dysglycemia

In another aspect, the present disclosure is directed to a methylation-specific polymerase chain reaction assay for determining dysglycemia in an obese adolescent subject suspected of having dysglycemia. The method includes isolating DNA from a sample obtained from the obese adolescent subject; treating the isolated DNA with bisulfite; amplifying methylated preproinsulin (INS) DNA in a sample obtained from the obese adolescent subject suspected; amplifying the preproinsulin (INS) promoter; and detecting whether a nucleotide located at position −69 from the preproinsulin (INS) transcriptional start site is methylated or unmethylated; comparing the concentration of methylated preproinsulin (INS) DNA in the sample obtained from the obese adolescent subject with the concentration of methylated preproinsulin (INS) DNA in an adolescent control subject selected from the group consisting of an adolescent control subject having normal weight, an adolescent control subject having normal glucose tolerance, and an adolescent having normal weight and normal glucose tolerance; and determining that the obese adolescent subject has dysglycemia when the concentration of methylated preproinsulin (INS) DNA in the sample is greater than the concentration of methylated preproinsulin (INS) DNA in the adolescent control subject.

In one embodiment, the obese adolescent subject has normal glucose tolerance. In another embodiment, the obese adolescent subject has glucose tolerance impairment.

A Methylation-Specific Polymerase Chain Reaction Assay for Determining Dysglycemia in an Adolescent Subject Having Type 2 Diabetes and Suspected of Having Dysglycemia

In another aspect, the present disclosure is directed to a methylation-specific polymerase chain reaction assay for determining dysglycemia in an obese adolescent subject having type 2 diabetes and suspected of having dysglycemia. The method includes isolating DNA from a sample obtained from the obese adolescent subject; treating the isolated DNA with bisulfite; amplifying methylated preproinsulin (INS) DNA in a sample obtained from the obese adolescent subject; amplifying the preproinsulin (INS) promoter; and detecting whether a nucleotide located at position −69 from the preproinsulin (INS) transcriptional start site is methylated or unmethylated; comparing the concentration of methylated preproinsulin (INS) DNA in the sample obtained from the obese adolescent subject with the concentration of methylated preproinsulin (INS) DNA in an adolescent control subject selected from the group consisting of an adolescent control subject having normal weight, an adolescent control subject having normal glucose tolerance, and an adolescent having normal weight and normal glucose tolerance; and determining that the obese adolescent subject has dysglycemia when the concentration of methylated preproinsulin (INS) DNA is greater than the concentration of methylated preproinsulin (INS) DNA in the adolescent control subject.

In another embodiment, the obese adolescent subject has type 2 diabetes and islet autoantibodies.

In another embodiment, the method further includes amplifying unmethylated preproinsulin (INS) DNA in a sample obtained from the obese adolescent subject; amplifying the preproinsulin (INS) promoter; and detecting whether a nucleotide located at position −69 from the preproinsulin (INS) transcriptional start site is methylated or unmethylated; comparing the concentration of unmethylated preproinsulin (INS) DNA in the sample obtained from the obese adolescent subject with the concentration of unmethylated preproinsulin (INS) DNA in an adolescent control subject selected from the group consisting of an adolescent control subject having normal weight, an adolescent control subject having normal glucose tolerance, and an adolescent having normal weight and normal glucose tolerance; and determining that the obese adolescent subject has dysglycemia when the concentration of unmethylated preproinsulin (INS) DNA is greater than the concentration of unmethylated preproinsulin (INS) DNA in the adolescent control subject.

Various functions and advantages of these and other embodiments of the present disclosure will be more fully understood from the examples described below. The following examples are intended to illustrate the benefits of the present disclosure, but do not exemplify the full scope of the disclosure.

### EXAMPLES

### Materials and Methods:

Human Subjects and Islets

After Institutional Review Board approval, banked serum samples were obtained from pediatric T1D subjects at disease onset (initial presentation) at Riley Hospital for Children. Subjects were 5-15 years of age and did not present in diabetic ketoacidosis (bicarbonate levels were greater than 15 mM and pH was greater than 7.3 in all individuals). Additionally, longitudinally collected banked serum samples were obtained from a subset of the same cohort at 8 weeks and 1 year after T1D onset. Banked serum from healthy pediatric subjects, lean adults without diabetes, obese adults without diabetes, adults with T2D (duration of disease 7.1±1.1 years) and adults with autoimmune hepatitis were obtained for use as comparisons. Age, gender, BMI Z-score for pediatric subjects or BMI for adult subjects, hemoglobin A1c, and C-peptide values were obtained from previously collected chart review data. Human islets were obtained from the Integrated Islet Distribution Program.

Animals

Immunocompetent CD1 mice were purchased from Charles River. All mice were maintained under protocols approved by the Indiana University School of Medicine Institutional Animal Care and Use Committee. Mice were fed regular chow diet and water ad libitum. For xenogeneic islet transplantation, 200 human islets were transplanted under the renal capsule of CD1 mice as described in Oh E, Stull N D, Mirmira R G, Thurmond D C (2014) Syntaxin 4 Up-Regulation Increases Efficiency of Insulin Release in Pancreatic Islets From Humans With and Without Type 2 Diabetes Mellitus. J Clin Endocrinol Metab 99:E866-E870. doi: 10.1210/jc.2013-2221. A separate group of control mice did not receive transplanted islets. Blood was obtained from the tail vein and centrifuged at 5,000 rpm for 10 minutes to isolate serum for DNA recovery. Blood was collected prior to transplantation and at 6 hours, 24 hours, 48 hours, and 7 days after transplantation, after which the animals were euthanized and kidneys were harvested.

DNA Extraction and Bisulfite Treatment

DNA was extracted from human islets using the genomic DNA extraction kit (Sigma-Aldrich). DNA was extracted from approximately 20 μl of mouse serum and 30-50 μl of human serum using the ZR serum DNA kit (Zymo Research Corporation, Irvine, Calif.) or the QIAamp DNA blood mini kit (Qiagen) with 5 μg of poly-A DNA as carrier. DNA recovery was ˜85%, with <10-15% variance between samples. All DNA samples then underwent bisulfite conversion using the EZ DNA Methylation Kit or the EZ DNA Methylation-Lightning Kit (Zymo Research, Irvine, Calif.), and conversion was verified using a pre- and post-conversion sample in the ddPCR.

PCR Analysis

Each sample was analyzed by ddPCR utilizing a custom designed dual fluorescent probe-based multiplex assay. For amplification of the human INS promoter, the following primers were used: 5′-GGAAATTGTAGTTTTAGTTTTTAGTTATTTGT-3′ (forward) (SEQ ID NO:1); 5′-AAAACCCATCTCCCCTACCTATCA-3′ (reverse) (SEQ ID NO:2) in combination with the following probes that detected methylation or unmethylation at position −69 relative to the transcriptional start site: 5′-ACCCCTACCGCCTAAC-3′ (VIC)—methylated (SEQ ID NO:3); 5′-ACCCCTACCACCTAAC-3′ (FAM)—unmethylated (SEQ ID NO:4). Primers and probes for mouse INS2 DNA are as follows: primers used included 5′-AATTGGTTTATTAGGTTATTAGGGTTTTTTGTTAAGATTTTA-3′ (forward) (SEQ ID NO:5); 5′-ACTAAAACTACAATTTCCAAACACTTCCCTAA-3′ (reverse) (SEQ ID NO:6); probes used included: 5′-CTCATTAAACGTCAACACC-3′(VIC) (SEQ ID NO:7); 5′-CTCATTAAACATCAACACC-3′ (FAM) (SEQ ID NO:8).

The PCR was performed using ddPCR Supermix for Probes (No dUTP) (Bio-Rad Laboratories, Inc., Hercules, Calif.) with the following cycling conditions: 95° C. for 10 minutes, 94° C. for 30 seconds, 57.5° C. for 60 seconds for 40 amplification cycles. Droplets were analyzed by the QX200 Droplet Reader and QuantaSoft Software (Bio-Rad Laboratories, Inc., Hercules, Calif.), from which an absolute concentration (copies/μl) of methylated and unmethylated INS DNA was obtained in each subject's sample. This final concentration was extrapolated to copies of unmethylated or methylated INS DNA/μl serum, then log-transformed for parametric statistical analysis.

Plasmid Standards

Amplified human INS PCR products from bisulfite-treated human islet DNA were subcloned into the T/A cloning vector pCR2.1 (Invitrogen, Grand Island, NY) and a minimum of 10 clones was sequenced to confirm the identity of the PCR products. Methylation- and unmethylation-specific plasmids were generated from the cloned PCR products to create standard PCR curves.

Statistical Analysis

For direct comparisons of methylation- and unmethylation-specific INS DNA levels, a two-tailed unpaired student's t-test was used. For comparison of longitudinally collected samples a two-tailed paired student's t-test was used. A p-value <0.05 was considered significant. All statistical calculations were done using PRISM™ 5.0 Software (GraphPad).

### Example 1

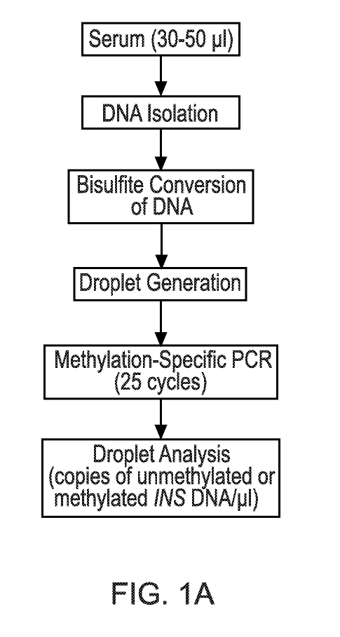

In this Example, a methylation-specific PCR (MSP) assay was developed to simultaneously quantitate methylation or unmethylation at the CpG at INS position −69 bp, shown in prior studies to be preferentially unmethylated in (3-cells. Control plasmids containing bisulfite-converted methylated or unmethylated INS DNA were used to standardize the MSP assay in ddPCR. FIG. 1E shows the gating strategy to distinguish methylated, unmethylated, and double-positive INS-containing droplets.

To verify linearity and ability to distinguish simultaneous mixtures of the DNA species, mixtures of plasmids at varying ratios were subjected to ddPCR, as shown in FIGS. 1B & 1C. These methylation specific plasmids were used to construct linearity curves over the range of DNA copy numbers observed in serum (FIG. 1D).

### Example 2

In this Example, the MSP assay of the present disclosure was used to detect dying human β cells in vivo.

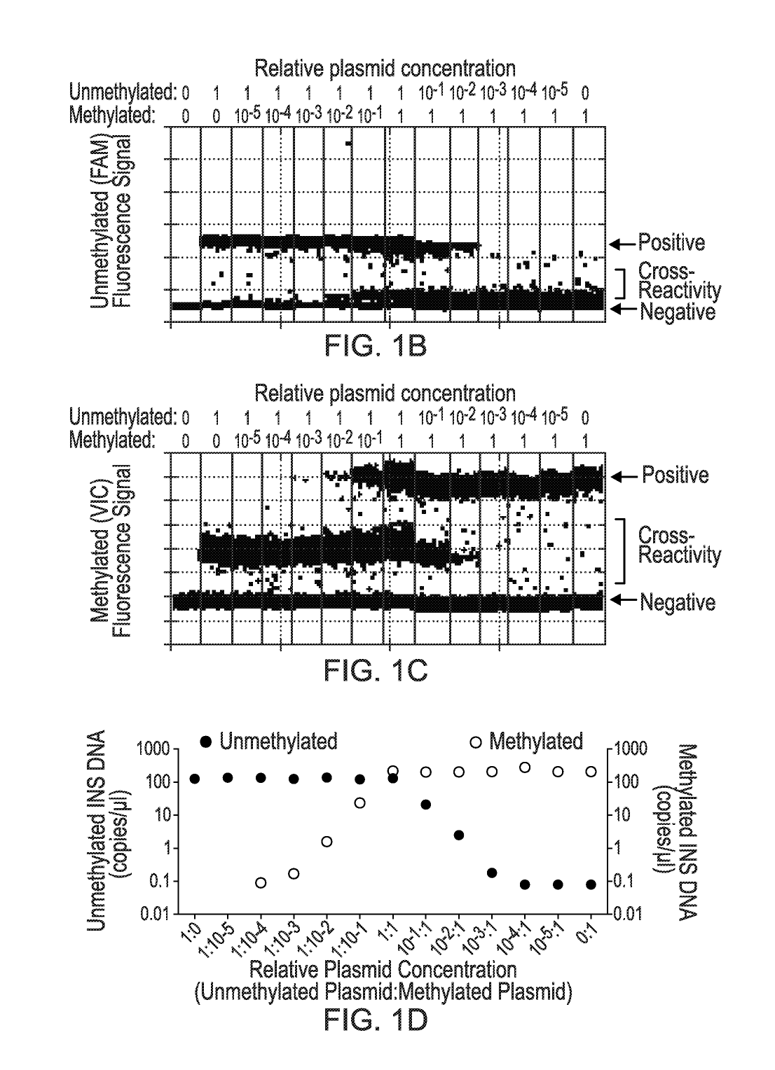

200 donor human islets were transplanted beneath the kidney capsule of healthy immunocompetent CD1 mice, and allowed to undergo xeno-rejection. Serum samples were collected prior to transplantation (time 0) and longitudinally at 6 hours, 24 hours, 48 hours, and 7 days after transplantation. Circulating unmethylated human INS peaked in the serum at 6 hours, falling to undetectable levels by 48 hours post-transplantation (FIG. 2A). By contrast, only a slight (insignificant) increase in methylated INS was detectable at 6 hours post transplantation (FIG. 2B). Upon removal of the kidney 7 days after transplantation, human islets were unidentifiable, suggesting that islets were completely rejected and killed (not shown). Neither unmethylated nor methylated human INS was measurable in mice that did not receive transplants, and neither unmethylated nor methylated mouse INS2 was changed in mice that did receive transplants (FIG. 2B). Collectively, these data indicate that rejection (death) of human islets in a xenotransplantation model is detectable primarily as an increase in unmethylated human INS in serum.

The MSP assay was further used in a mouse model of autoimmune (3-cell destruction (NOD mice). Compared to NOD-SCID and CD1 controls, NOD mice exhibited elevated levels of both unmethylated and methylated mouse INS2 in the pre-diabetic phase, with levels falling at the time of diabetes (FIGS. 2C & 2D).

### Example 3

In this Example, the utility of the MSP assay of the present disclosure was analyzed for detection of β cell stress/death in subjects with new-onset T1D.

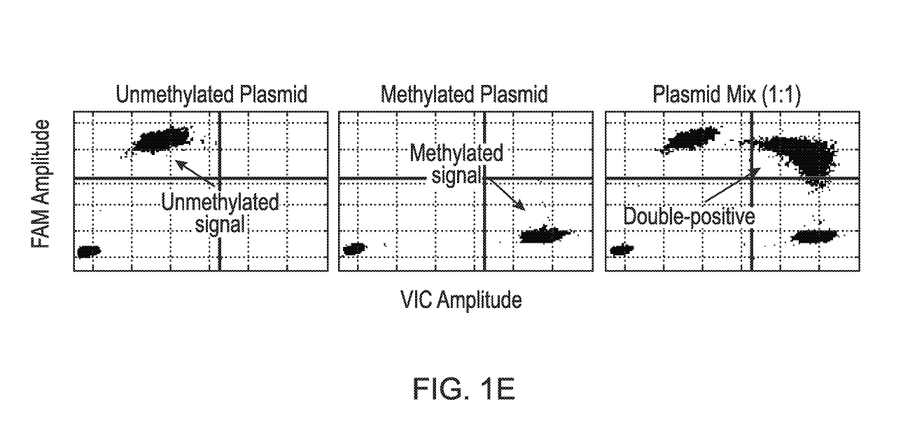

Serum samples were obtained from 32 pediatric subjects with T1D within two days of diabetes diagnosis (a timeframe presumed to be at or near onset of disease). Additionally, 24 of these subjects had a subsequent serum sample collected 8 weeks post diagnosis and 8 of these subjects had serum collected approximately 1 year post diagnosis. Relevant demographic and laboratory data of all subjects are presented in Table 1, and representative 2-dimensional and 1-dimensional plots are shown in FIGS. 3A-3D. Notably, Hgb A1c values significantly decreased and C-peptide levels increased 8 weeks after diabetes onset (p<0.0001 and p=0.0002 respectively). For comparison, serum samples were also collected from 27 healthy pediatric control subjects (Table 1).

TABLE 1
Demographic and Laboratory Evaluation of Subject Cohorts

| | | | | | Lean | Obese | | Adult |
|---|---|---|---|---|---|---|---|---|
| | Pediatric | T1D at | T1D at | T1D at | Adult | Adult | Adult | Autoimmune |
| | Control | diagnosis | 8 weeks | 1 Year | Control | Control | T2D | Hepatitis |
| Age (yrs) | 9.5 ± 3.6 | 10.8 ± 3.0 | 10.7 ± 3.1 | 11.6 ± 3.1 | 51.3 ± 9.0 | 49.3 ± 5.6 | 48.6 ± 7.5 | 47 ± 14 |
| Female/Male | 14/13 | 14/18 | 11/13 | 1/7 | 12/3 | 11/10 | 5/12 | 12/2 |
| BMI Z-Score | −0.08 ± 0.7 | −0.22 ± 1.3 | +0.54 ± 0.9 | | | | | |
| BMI | | | | | 22.9 ± 1.2 | 32.7 ± 3.5 | 35.9 ± 7.4 | 30.5 ± 7.7 |
| Hgb A1c | | 11.3 ± 1.7 | 7.6 ± 0.8 | 8.3 ± 1.4 | | | 8.4 ± 1.6 | |
| (%) | | | | | | | | |
| C-Peptide | | 150 ± 167 | 303 ± 166 | | | | | |
| (pM) | | | | | | | | |

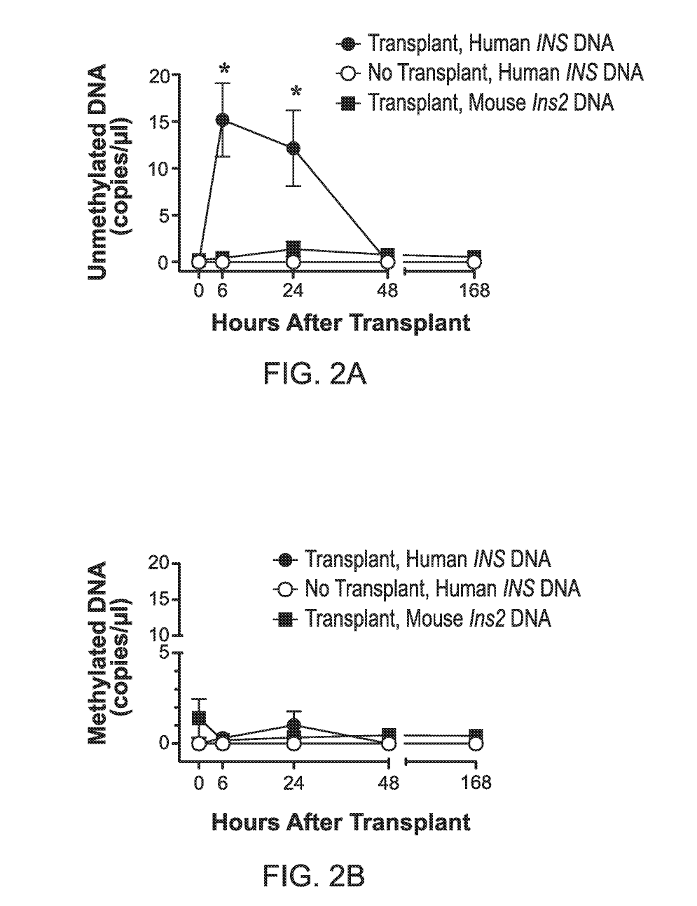

As shown in FIGS. 4A and 4C, levels of both unmethylated and methylated circulating INS were significantly higher in pediatric subjects at T1D onset compared to healthy pediatric controls (p<0.0001). At 8 weeks following onset of T1D, the levels of unmethylated INS decreased significantly (p<0.0001) and were statistically no different than controls (FIGS. 4A and B). By contrast, levels of methylated INS remained elevated 8 weeks after T1D onset compared to controls (p<0.0001). At one year post T1D diagnosis, methylated INS levels showed reductions (FIG. 4D), and were in fact lower than controls (p=0.02, FIG. 4C). Unmethylated INS levels remained at the same levels at 1 year post T1D diagnosis as at 8 weeks post diagnosis, but were still higher than controls (p<0.0001) (FIGS. 4A and B). There were no correlations of unmethylated or methylated circulating insulin levels with Hgb A1c or C-peptide levels at onset (data not shown).

Whereas circulating unmethylated INS is thought to arise primarily from stressed/dying islet β cells, circulating methylated INS could arise from virtually any stressed/dying cell type. Therefore, the elevated levels of methylated INS observed in T1D subjects were further analyzed to determine if they reflect a generalized cellular response to either autoimmunity or prevailing hyperglycemia. To this end, MSP assays were performed using serum from adults with active (biopsy-verified) autoimmune hepatitis, from adults with type 2 diabetes (T2D), and from lean and obese healthy adult controls (see demographics in Table 1). Methylated INS levels were no different between lean and obese adults and unmethylated INS levels were slightly lower, but with significant overlap, in the obese controls (p=0.04) (FIGS. 4A and 4C). Methylated and unmethylated INS in both of these adult control groups were higher than pediatric controls, suggesting that these circulating DNA species may exhibit age-related differences (FIGS. 4A and C). Compared to both healthy adult control groups, unmethylated and methylated circulating INS levels were either no different or lower in subjects with autoimmune hepatitis or T2D (FIGS. 4A and C). Notably, those with autoimmune hepatitis had significantly lower levels of unmethylated and methylated INS compared to subjects with T1D (p<0.0001) (FIGS. 4A and C). Additionally, levels of circulating unmethylated and methylated INS were lower in T2D subjects than in T1D subjects at diagnosis (p=0.04 and p<0.0001, respectively) (FIGS. 4A and C). Collectively, these data suggest that, unlike T1D, elevations in unmethylated and methylated INS are not observed in T2D and a related autoimmune disease.

The above data reveal three important new findings: (1) unmethylated INS is increased at T1D diagnosis, and falls to near control levels by 8 weeks post-diagnosis, (2) methylated INS is elevated at T1D diagnosis and at 8 weeks post-diagnosis, and falls by 1 year post-diagnosis, and (3) elevations in both methylated and unmethylated INS appear to be specific for new-onset T1D, since concomitant elevation of both species is not observed at any subsequent time point in T1D or in other disorders of immunity or glycemia (autoimmune hepatitis or T2D).

An unexpected finding in the Examples above is the elevation of methylated INS in subjects with T1D. Whereas unmethylated INS is thought to arise primarily from islet β cells, methylated INS could arise from virtually any cell type. The present disclosure, therefore, provides unique patterns of circulating methylated and unmethylated INS that can not only be specific for T1D, but can also be relevant to different stages of disease progression. In conclusion, the present disclosure emphasizes the need to examine absolute copy numbers of DNA species to utilize cell-free DNA species as biomarkers of disease.

### Example 4

In this Example, differentially methylated multiple CpG sites in β cells and non-β cell were identified.

Specifically, a methylation specific Infinium HumanMethylation450 array of 64 human islet preparations versus 27 human non-β cell tissues/cell lines was analyzed. Informatics analysis of these datasets identified 2534 hypomethylated CpG sites and 3667 hypermethylated CpG sites in human islets vs. non-islet tissues. Notwithstanding the impact of methylation on gene expression, only 8 β cell-specific genes (representing fewer than 100 of the CpG sites) showed differential methylation, meaning that the majority of differentially methylated CpG sites did not occur in differentially methylated genes.

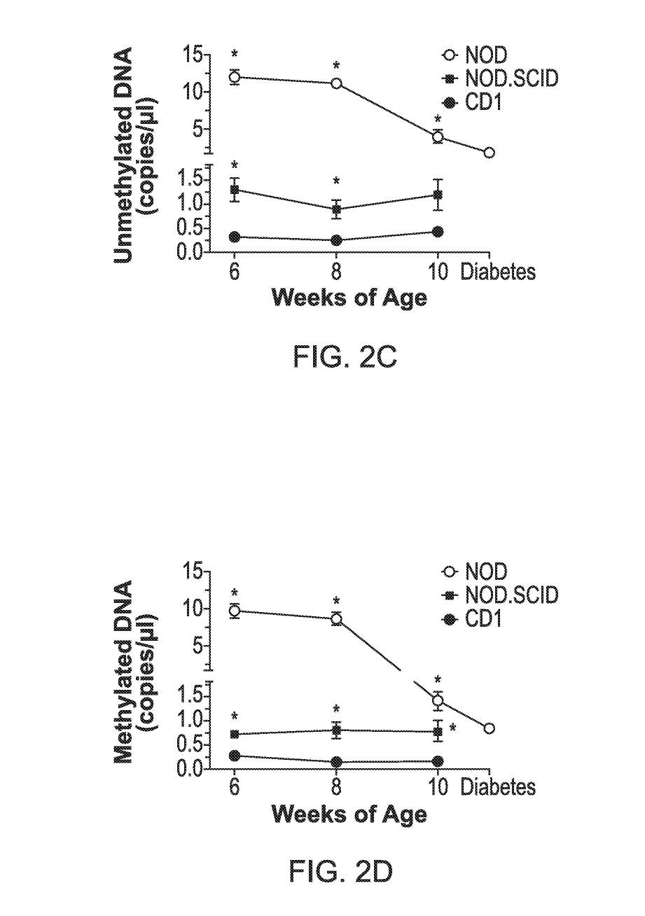

FIG. 5A depicts the expression heat map of the differently methylated CpG sites in human islets vs. non-islet tissues (none of which were found in differentially expressed genes). FIG. 5B depicts the 10 most highly differently methylated CpG sites including: chr3: 125085322 (ZNF148, zinc finger protein 148); chr3:12586368 (intergenic); ch1:153610672 (Clorf77, chromosome 1, open reading frame 77), chr3: 135702110 (PPP2R3A; Serine/threonine-protein phosphatase 2A regulatory subunit B); chr2:189064557 (intergenic); chr14:105491309 (intergenic); chr5:35046992 (AGXT2, alanine-glyoxylate aminotransferase 2); chr7:107300287 (SLC26A4, Solute Carrier Family 26 (Anion Exchanger), Member 4); chr8:126649807 (intergenic); and chr12:49759545 (SPATS2, spermatogenesis associated, serine rich 2).

### Example 5

In this Example, differentially methylated β cell specific DNA were determined in urine.

The ability to use urine is advantageous because it is obtained non-invasively, it is easier to obtain more frequently, it can be obtained in larger volumes, and offers a potentially more integrative result because it can be collected over longer periods of time than serum. To determine if changes in methylated and unmethylated INS DNA could be detected in urine, DNA was isolated from a new-onset T1D subject (within 48 h of diagnosis) and an age-matched control subject. ddPCR was performed as described.

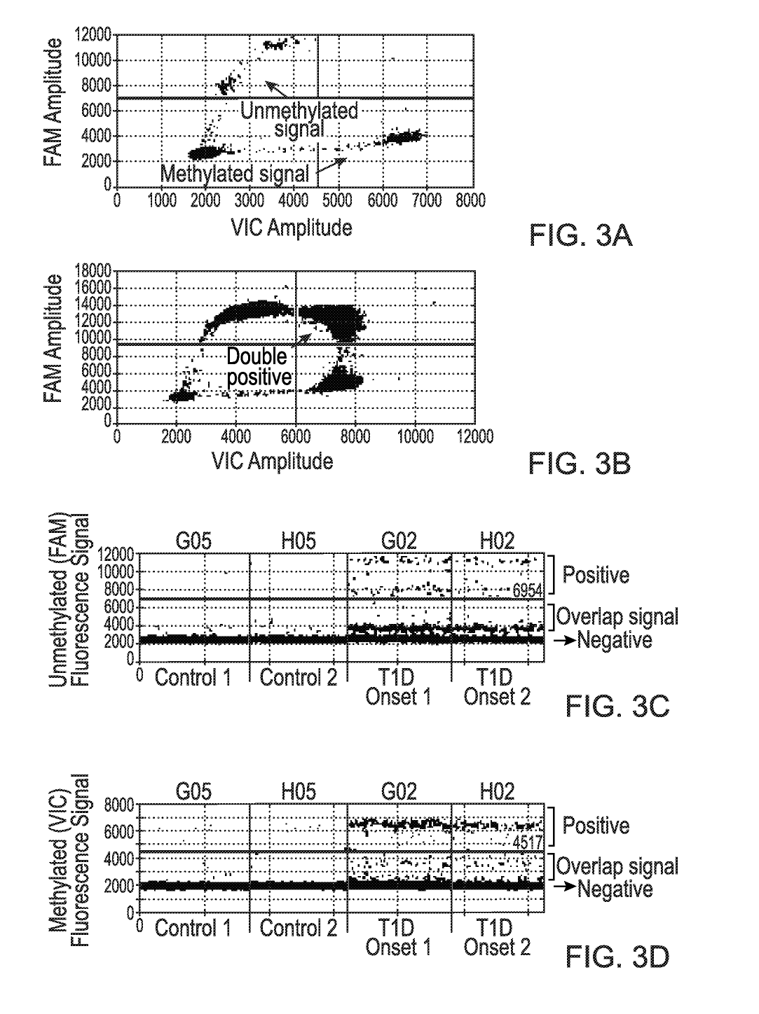

FIG. 6 shows that both methylated and unmethylated INS DNA (at position −69 bp) were elevated in the new-onset subject compared to the control. These results demonstrate that differentially methylated DNA can be detected in urine samples.

### Example 6

In this Example, a longitudinal study determining β cell death by methylation status of the INS gene was used in individuals with glucose intolerance and T2D.

Human Subjects. Serum samples were obtained from male and female adult subjects 18-65 years of age. After an overnight fast, subjects underwent an oral glucose tolerance test (OGTT) with 75 g Glucola. Subjects were divided into three categories based on 2 hour OGTT: normal glucose tolerance (NGT; <140 mg/dL), impaired glucose tolerance (IGT; 140-199 mg/dL), and those with diabetes (T2D; >200 mg/dL). Diabetes medications were held for 48 hours prior to data/sample collection. Exclusion criteria included: Metformin use 4 weeks previous, Thiazolidinediones use 6 months previous, T1D, other diabetes, pregnancy, weight fluctuation 6 months previous, current or past tobacco use, acute or chronic illness, pulmonary disease, or use of antidepressants. Participants were provided written informed consent for screening and continuing study participation. The study was approved by the Indiana University Institutional Review Board.

Animal Studies. Male C57B1/6J (BL6) were obtained from The Jackson Laboratory and maintained under protocols approved by the Indiana University School of Medicine Institutional Animal Care and Use Committee. All mice were kept under pathogen-free conditions with a standard light-dark cycle and received water ad libitum.

BL6 mice were acclimated for 1 week prior to being placed on either a low fat diet (10% kCAL from fat, Research Diets; D12450B) or high fat diet (60% kCal from fat; Research Diets; D12492) starting at 8 weeks of age. Blood was harvested by tail vein weekly and processed to plasma. Body weights were measured weekly and fasting blood glucose was measured via tail vein every 2 weeks. After 10 weeks of diet treatment, mice received multiple low dose streptozocin (MLDS-STZ). Five I.P. injections of 55 mg/dl STZ was given daily. A subset of mice from each group was euthanized each week for β cell mass measurements. 14 weeks post start of the diet, mice were euthanized and pancreas was harvested.

DNA extraction, bisulfite treatment and ddPCR. DNA was extracted from 20 μl (mouse) or 50 μl (human) of serum/plasma samples using the QIAamp Blood DNA Mini Kit (Qiagen). All DNA samples underwent bisulfite treatment using the EZ-Lightening DNA Methylation kit (Zymo Research).

Samples were analyzed by ddPCR utilizing a dual fluorescent probe-based multiplex assay. Primers and probes for mouse Ins2 and human INS DNA were described previously (Marisa M. Fisher et al. 2013; Marisa M. Fisher et al. 2015). Droplets were analyzed by a QX200 Droplet Reader and QuantaSoft Software (Bio-Rad), from which concentrations (copies/μl) of methylated and unmethylated INS DNA were obtained. This final concentration was extrapolated to copies/μl serum, then log-transformed for parametric statistical analysis.

Immunostaining and morphometric assessment of β cell area. Pancreata from at least three different mice per group were fixed in 4% paraformaldehyde, paraffin embedded, and sectioned onto glass slides. The β cell area as a percentage of total pancreas area (β cell area%) was calculated as previously detailed using a Zeiss Axio Scan.Z1 (Maier et al. 2010).

Statistical Analysis. All data are presented as mean +/− SEM. A Student's t test was performed for all comparisons involving two conditions. For comparisons of more than two variables, a one-way ANOVA with a Tukey post-test was performed. Prism 6 software (GraphPad) was used for all statistical tests. Statistical significance was assumed at P <0.05.

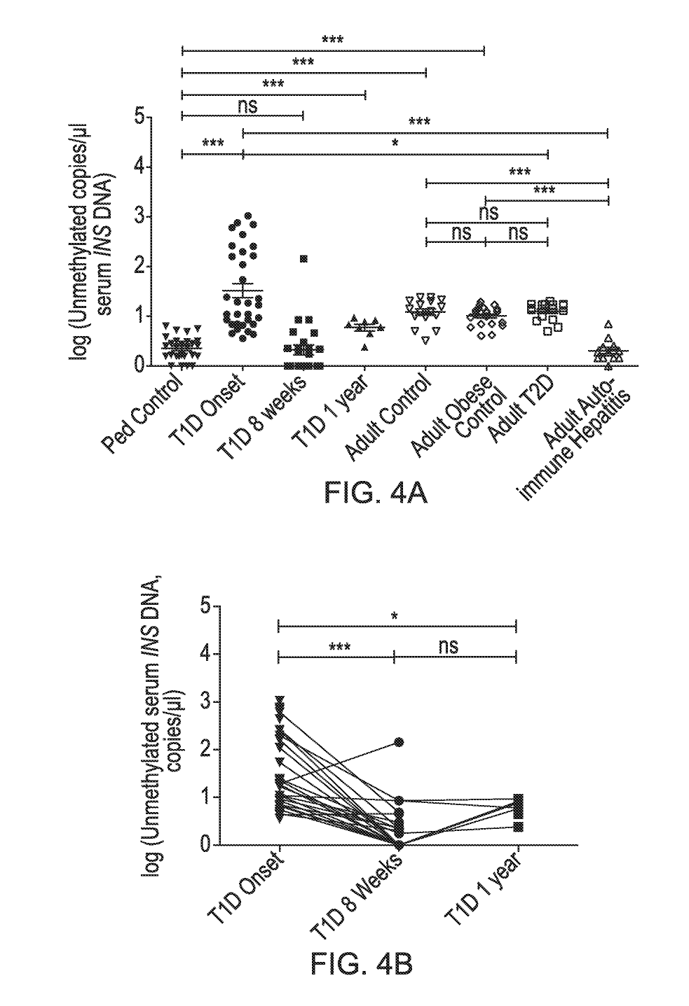

No increases in unmethylated INS DNA in cell-free DNA isolated from serum was observed in subjects with T2D (FIG. 7A) Likewise subjects with impaired glucose tolerance (IGT) as measured by oral glucose tolerance test had similar levels of unmethylated INS DNA compared to subjects with normal glucose tolerance (NGT). Subjects with T2D also had similar levels of methylated INS DNA compared to NGT subjects; however, subjects with IGT had slight, but significantly elevated methylated INS DNA compared to NGT subjects (FIG. 7B). These data indicate that β cell death may be occurring prior to overt T2D and that β cell death may not be readily detectable in cross-sectional sampling. In order to determine if β cell death could be captured using longitudinal sampling the methylation status of mouse Ins2 DNA was measured in serum samples from a mouse model of prediabetes.

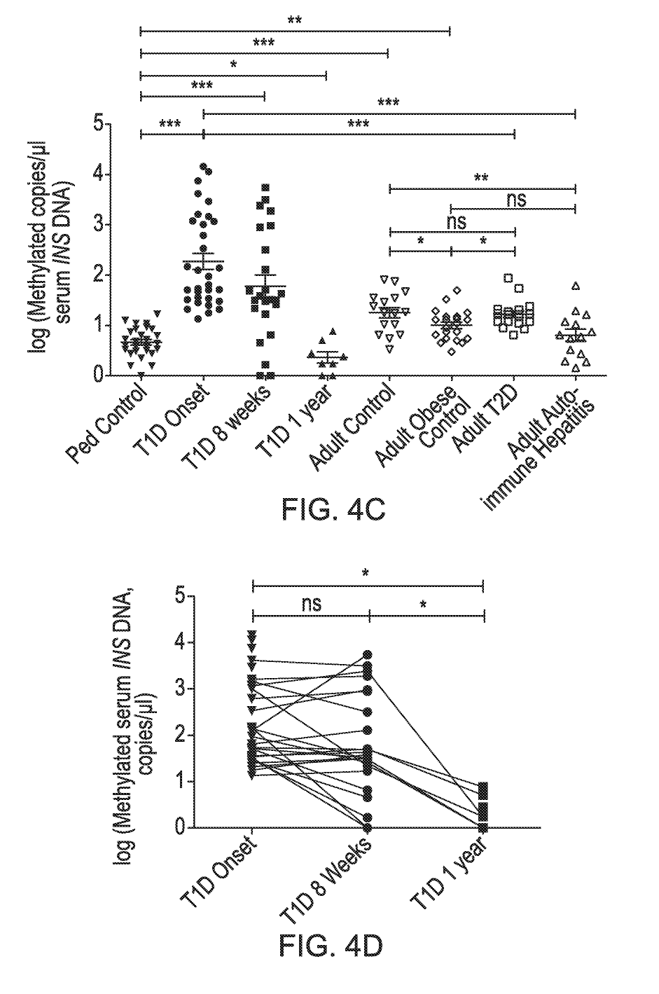

Prior to analyzing the methylation status of Ins2 DNA in HFD-fed mice, the assay was validated using DD-PCR of the -182 site of Ins2 in the mouse DNA previously described to be differently methylated in β cells (Marisa M. Fisher et al. 2013; Kuroda et al. 2009b). Using MIN6 β cells, DNA was isolated and bisulfite treated prior to subcloning into a plasmid. Because MIN6 β cells have both unmethylated and methylated Ins2 DNA, both an unmethylated and a methylated plasmid were able to be generated, as verified by sequencing. As shown in FIGS. 8A and 8B, the mouse MSP assay was able to distinguish unmethylated Ins2 using the FAM-labeled probe and methylated Ins2using the VIC-labeled probe in a linear fashion.

Next, mouse islet DNA was isolated and added to mouse serum. The DNA-spiked serum was diluted and results showed further linearity for both unmethylated and methylated Ins2 DNA in the same sample (FIGS. 8C and 8D).

To examine β cell death longitudinally in a mouse model of prediabetes, BL6 mice were fed a high fat diet (HFD; 60% kCal from fat) starting at 8 weeks of age and compared to BL6 mice fed a low fat diet (LFD; 10% kCal from fat).

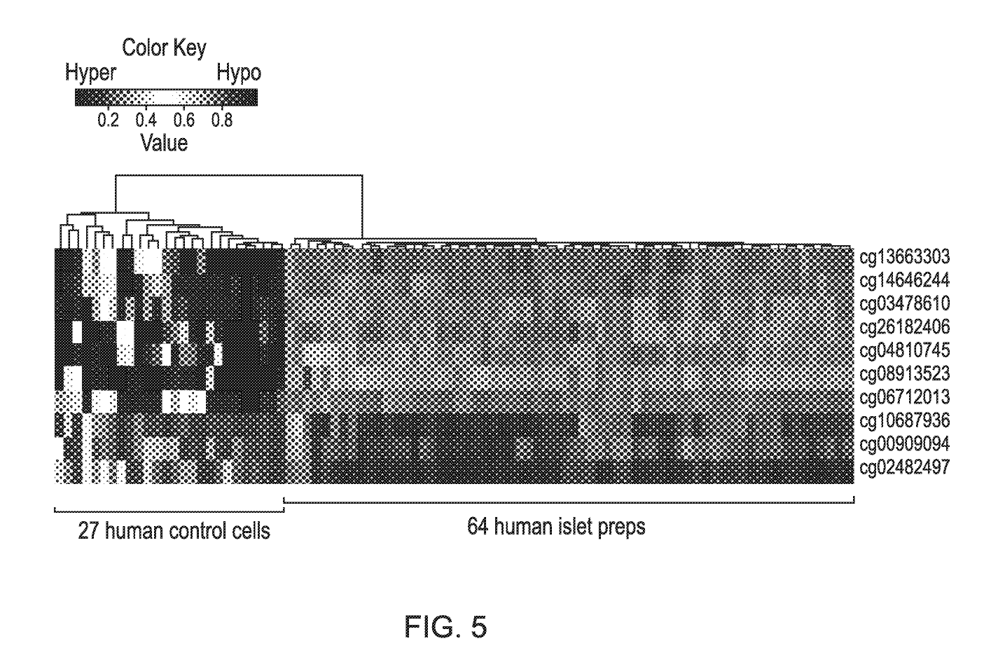

HFD-fed BL6 mice exhibited increased body weights and fasting blood glucose values by 6 weeks post start of diet (FIGS. 9A and 9B). Although fasting blood glucose values were not elevated until 6 weeks of age, HFD-fed mice showed impaired glucose tolerance by GTT as early as 2 weeks post start of diet (FIG. 9C). β cell mass of HFD-fed animals increased significantly by 6 weeks of age compared to LFD-fed animals (FIG. 9D).

β cell death was then examined using the MSP assay. Unmethylated Ins2 DNA was increased in HFD-fed mice specifically at 2 and 6 weeks of age, the same as the first signs of β cell dysfunction as measured by impaired glucose tolerance. Methylated Ins2 DNA was unchanged at all time points of HFD feeding. At 10 weeks of age, mice were given MLDS STZ to induce significant β cell death. As shown in FIGS. 9E and 9F, both unmethylated and methylated Ins2 DNA were significantly increased after STZ injections. These data indicate that β cell death occurs transiently in the beginning stages of prediabetes and prior to overt diabetes, and may not be readily detectable in cross-sectional studies.

The results described herein in demonstrate methylated and unmethylated DNA as a marker for TID in serum and urine samples. The results also demonstrate markers of β cell death may only be detectable before onset of T2D, and most readily demonstrated in longitudinal studies of animals or people at risk for progression to T2D.

### Example 7

In this Example, methylated and unmethylated preproinsulin (INS) DNA in adolescent subjects was analyzed for determining dysglycemia.

DNA was isolated from banked serum from obese normal glucose tolerance (NGT), impaired glucose tolerance (IGT) and type 2 diabetes (T2D) adults and youth, and assessed differentially methylated INS by droplet digital PCR. Blood was also analyzed using the A1c test for glycosylated hemoglobin.

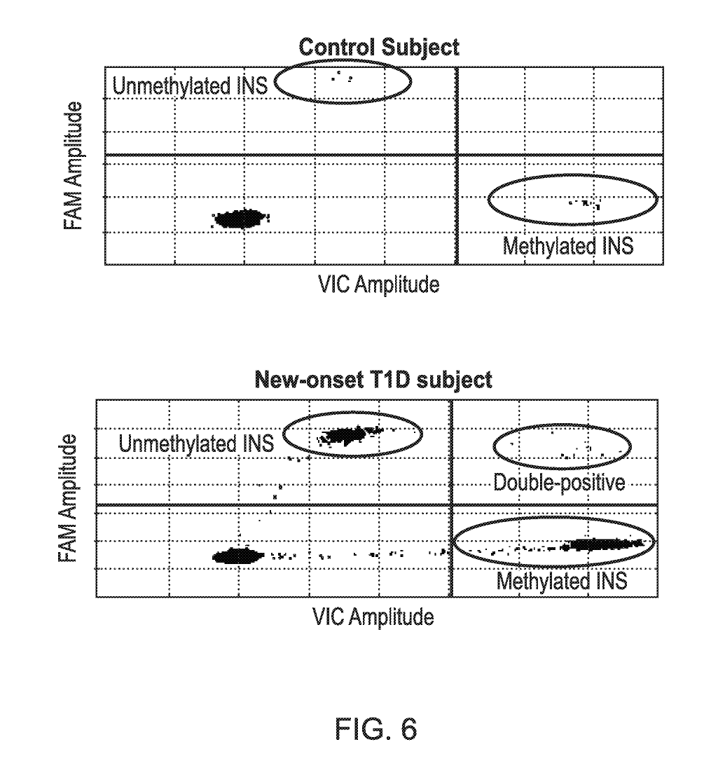

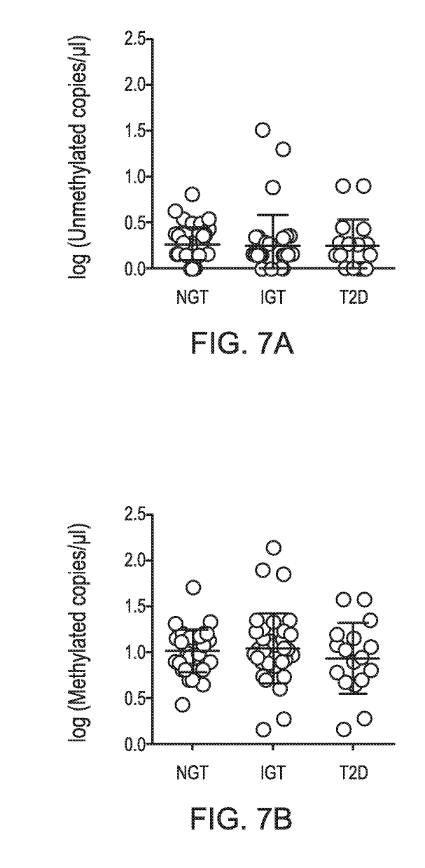

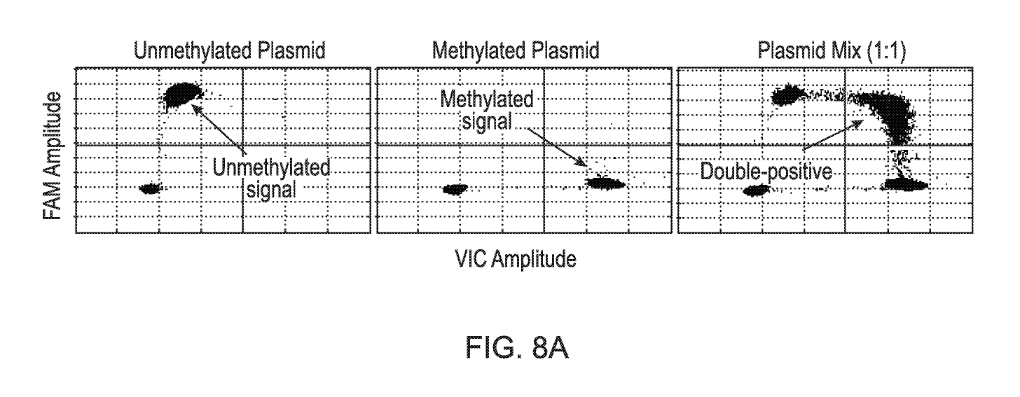

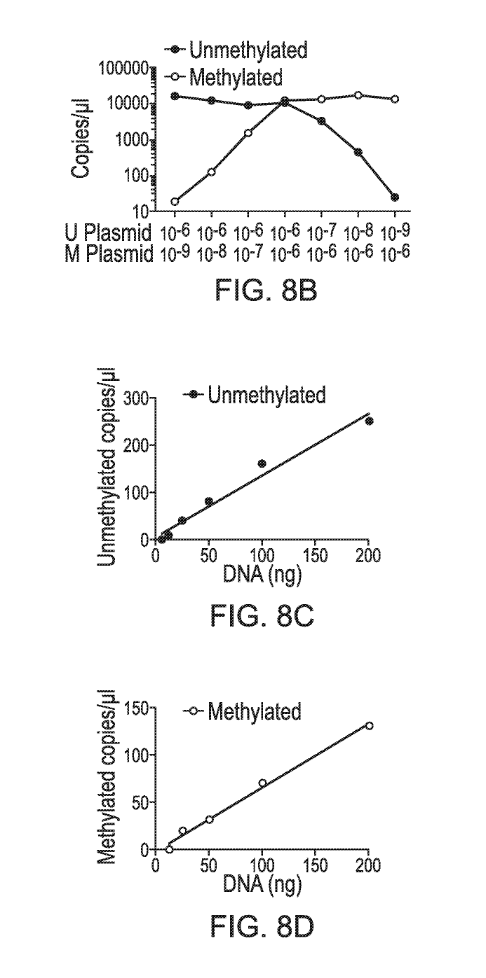

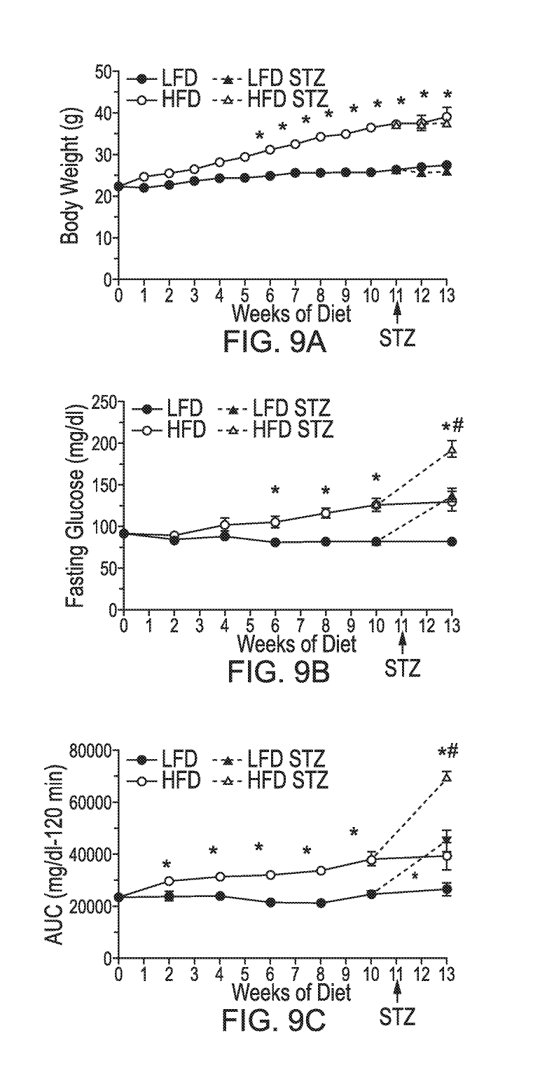

Among non-dysglycemic controls, values for both methylated and unmethylated INS increased with age (P<0.001, R=0.229; P<0.001, R=0.3201) (FIG. 10A and 10B), with adults showing 3-30-fold higher levels than youth (FIGS. 10A and 10B). Among adults, those with IGT and T2D exhibited no differences in unmethylated or methylated INS levels compared to controls (P>0.9) (FIG. 11A and 11B and 12A and 12B). Obese youth with NGT and IGT exhibited elevated levels of methylated INS compared to normal weight NGT controls (P<0.001; P<0.01), but showed no differences in unmethylated INS (FIGS. 13A and 13B and 14A and 14B).

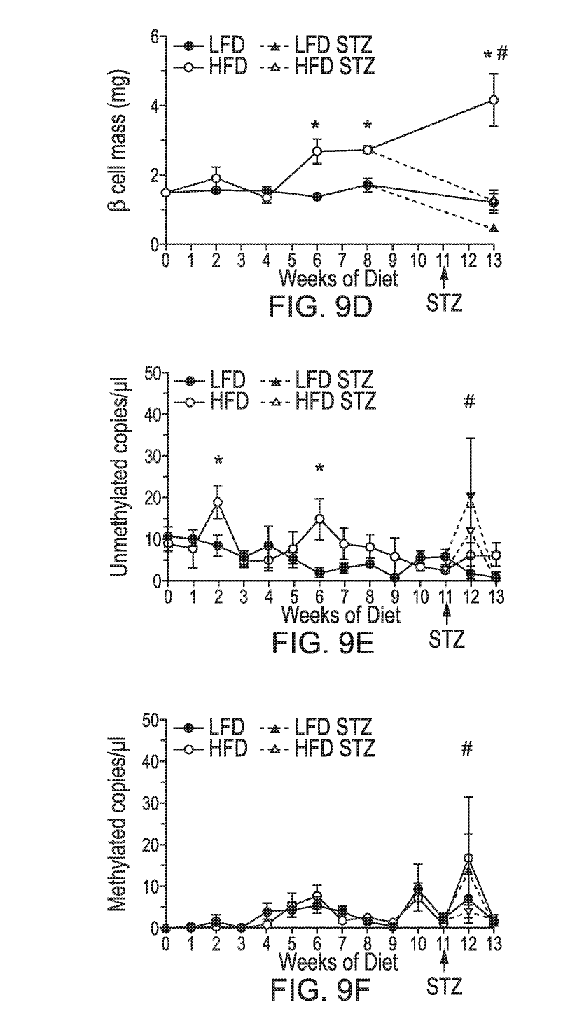

Youth with phenotypic T2D also showed elevated methylated INS levels compared to normal weight controls (P<0.01), and a trend to an increased unmethylated INS (P=0.059). The elevation in methylated INS levels in phenotypic T2D youth was driven by a subset with islet autoantibodies (P<0.001) (FIGS. 13A and 13B and 14A and 14B). Levels of methylated INS DNA also correlated with A1c in youth (FIGS. 15A and 15B).

These results demonstrate that while biomarkers coincident with β cell death are evident in youth with obesity, dysglycemia and diabetes, these markers are not elevated in adults with IGT and T2D. Whereas the different outcomes in adults and youth may reflect a superior signal-to-noise ratio for the assay in youth, they may also reflect accelerated β cell death in the pathogenesis of dysglycemia in childhood in contrast to adults in whom aging-associated β cell death might overshadow this process.

1-18. (canceled)

19. A method for determining new-onset type 1 diabetes in a subject suspected of having new-onset type 1 diabetes, the method comprising:
amplifying methylated preproinsulin (INS) DNA in a sample obtained from the subject suspected of having new-onset type 1 diabetes;
amplifying unmethylated preproinsulin (INS) DNA in a sample obtained from the subject suspected of having new-onset type 1 diabetes;
detecting whether a nucleotide located at position −69 from the preproinsulin (INS) transcriptional start site is methylated or unmethylated;
comparing the concentration of methylated preproinsulin (INS) DNA and unmethylated preproinsulin (INS) DNA in the sample with the concentration of methylated preproinsulin (INS) DNA and unmethylated preproinsulin (INS) DNA in a control; and
determining that the subject has new-onset type 1 diabetes when the concentration of methylated preproinsulin (INS) DNA and unmethylated preproinsulin (INS) DNA in the sample is greater than the concentration of methylated preproinsulin (INS) DNA and unmethylated preproinsulin (INS) DNA in the control.

20. The method of claim 19, wherein the nucleotide located at position −69 from the preproinsulin (INS) transcriptional start site is cytosine.

21. The method of claim 19, wherein the methylated preproinsulin (INS) DNA in the sample is amplified using an oligonucleotide comprising SEQ ID NO:3.

22. The method of claim 19, wherein the unmethylated preproinsulin (INS) DNA in the sample is amplified using an oligonucleotide comprising SEQ ID NO:4.

23. The method of claim 19 further comprising amplifying the preproinsulin (INS) promoter.

24. The method of claim 23, wherein the preproinsulin (INS) promoter is amplified using a first oligonucleotide comprising SEQ ID NO:1 and a second oligonucleotide comprising SEQ ID NO:2.

25. (canceled)

26. The method of claim 19, wherein the nucleotide located at position −69 from the preproinsulin (INS) transcriptional start site is methylated.

27. (canceled)

28. (canceled)

29. The method of claim 19, wherein the nucleotide located at position −69 from the preproinsulin (INS) transcriptional start site is unmethylated.

30. (canceled)

31. The method of claim 19, wherein the preproinsulin (INS) DNA in the sample is subjected to a bisulfite reaction.

32. The method of claim 19, wherein the sample is selected from the group consisting of serum, plasma, whole blood, and urine.

33.-37. (canceled)

38. A method of distinguishing between type 1 diabetes and type 2 diabetes in a subject suspected of having type 1 diabetes or type 2 diabetes, the method comprising
amplifying methylated preproinsulin (INS) DNA in a sample obtained from the subject suspected of having type 1 diabetes;
amplifying unmethylated preproinsulin (INS) DNA in a sample obtained from the subject suspected of having new-onset type 1 diabetes; and
detecting whether a nucleotide located at position −69 from the preproinsulin (INS) transcriptional start site is methylated or unmethylated;
comparing the concentration of methylated preproinsulin (INS) DNA and the concentration of unmethylated preproinsulin (INS) DNA from the subject to the concentration of methylated preproinsulin (INS) DNA and the concentration of unmethylated preproinsulin (INS) DNA from a subject having type 2 diabetes; and
determining that the subject has type 1 diabetes if the concentration of methylated preproinsulin (INS) DNA and the concentration of unmethylated preproinsulin (INS) DNA from the subject are elevated when compared to the concentration of methylated preproinsulin (INS) DNA and the concentration of unmethylated preproinsulin (INS) DNA of a subject having type 2 diabetes.

39. The method of claim 38, wherein a nucleotide located at position −69 from the preproinsulin (INS) transcriptional start site is cytosine.

40. The method of claim 38, wherein the methylated preproinsulin (INS) DNA in the sample is amplified using an oligonucleotide comprising SEQ ID NO:3 and the unmethylated preproinsulin (INS) DNA in the sample is amplified using an oligonucleotide comprising SEQ ID NO:4.

41. The method of claim 38 further comprising amplifying the preproinsulin (INS) promoter.

42. The method of claim 41, wherein the preproinsulin (INS) promoter is amplified using a first oligonucleotide comprising SEQ ID NO:1 and a second oligonucleotide comprising SEQ ID NO:2.

43. A method for diagnosing new-onset type 1 diabetes in a subject suspected of having new-onset type 1 diabetes, the method comprising:
amplifying methylated biomarker DNA in a sample obtained from the subject suspected of having new-onset type 1 diabetes;
amplifying unmethylated biomarker DNA in a sample obtained from the subject suspected of having new-onset type 1 diabetes;
comparing the concentrations of methylated biomarker DNA and unmethylated biomarker DNA in the sample with the concentrations of methylated biomarker DNA and unmethylated biomarker DNA in a control subject; and
determining that the subject has new-onset type 1 diabetes when the concentrations of methylated biomarker DNA and unmethylated biomarker DNA in the sample is greater than the concentrations of methylated biomarker DNA and unmethylated biomarker DNA in the control subject.

44. The method of claim 43, wherein the biomarker DNA is selected from the group consisting of preproinsulin; chr3: 125085322 (ZNF148, zinc finger protein 148); chr3:12586368 (intergenic); ch1:153610672 (C1orf77, chromosome 1, open reading frame 77), chr3: 135702110 (PPP2R3A; Serine/threonine-protein phosphatase 2A regulatory subunit B); chr2:189064557 (intergenic); chr14:105491309 (intergenic);
chr5:35046992 (AGXT2, alanine-glyoxylate aminotransferase 2); chr7:107300287 (SLC26A4, Solute Carrier Family 26 (Anion Exchanger), Member 4); chr8:126649807 (intergenic); chr12:49759545 (SPATS2, spermatogenesis associated, serine rich 2); and
combinations thereof.

45. The method of claim 43, wherein the sample is selected from the group consisting of serum, plasma, whole blood, and urine.

46. The method of claim 43, wherein DNA in the sample is subjected to a bisulfite reaction.

47.-70. (canceled)


SEQUENCE LISTING (9 sequences)

SEQ ID NO: 1 - DNA, 32 bp, Artificial Sequence
  Synthetic
ggaaattgtagttttagtttttagttatttgt

SEQ ID NO: 2 - DNA, 24 bp, Artificial Sequence
  Synthetic
aaaacccatctcccctacctatca

SEQ ID NO: 3 - DNA, 16 bp, Artificial Sequence
  Synthetic
acccctaccgcctaac

SEQ ID NO: 4 - DNA, 16 bp, Artificial Sequence
  Synthetic
acccctaccacctaac

SEQ ID NO: 5 - DNA, 42 bp, Artificial Sequence
  Synthetic
aattggtttattaggttattagggttttttgttaagatttta

SEQ ID NO: 6 - DNA, 32 bp, Artificial Sequence
  Synthetic
actaaaactacaatttccaaacacttccctaa

SEQ ID NO: 7 - DNA, 19 bp, Artificial Sequence
  Synthetic
ctcattaaacgtcaacacc

SEQ ID NO: 8 - DNA, 19 bp, Artificial Sequence
  Synthetic
ctcattaaacatcaacacc

SEQ ID NO: 9 - DNA, 4992 bp, Homo sapiens
ctcgaggggcctagacattgccctccagagagagcacccaacaccctccaggcttgaccggccagggtgtccccttcctaccttggagagagcagccccagggcatcctgcagggggtgctgggacaccagctggccttcaaggtctctgcctccctccagccaccccactacacgctgctgggatcctggatctcagctccctggccgacaacactggcaaactcctactcatccacgaaggccctcctgggcatggtggtccttcccagcctggcagtctgttcctcacacaccttgttagtgcccagcccctgaggttgcagctgggggtgtctctgaagggctgtgagcccccaggaagccctggggaagtgcctgccttgcctccccccggccctgccagcgcctggctctgccctcctacctgggctccccccatccagcctccctccctacacactcctctcaaggaggcacccatgtcctctccagctgccgggcctcagagcactgtggcgtcctggggcagccaccgcatgtcctgctgtggcatggctcagggtggaaagggcggaagggaggggtcctgcagatagctggtgcccactaccaaacccgctcggggcaggagagccaaaggctgggtgtgtgcagagcggccccgagaggttccgaggctgaggccagggtgggacatagggatgcgaggggccggggcacaggatactccaacctgcctgcccccatggtctcatcctcctgcttctgggacctcctgatcctgcccctggtgctaagaggcaggtaaggggctgcaggcagcagggctcggagcccatgccccctcaccatgggtcaggctggacctccaggtgcctgttctggggagctgggagggccggaggggtgtaccccaggggctcagcccagatgacactatgggggtgatggtgtcatgggacctggccaggagaggggagatgggctcccagaagaggagtgggggctgagagggtgcctggggggccaggacggagctgggccagtgcacagcttcccacacctgcccacccccagagtcctgccgccacccccagatcacacggaagatgaggtccgagtggcctgctgaggacttgctgcttgtccccaggtccccaggtcatgccctccttctgccaccctggggagctgagggcctcagctggggctgctgtcctaaggcagggtgggaactaggcagccagcagggaggggacccctccctcactcccactctcccacccccaccaccttggcccatccatggcggcatcttgggccatccgggactggggacaggggtcctggggacaggggtccggggacagggtcctggggacaggggtgtggggacaggggtctggggacaggggtgtggggacaggggtgtggggacaggggtctggggacaggggtgtggggacaggggtccggggacaggggtgtggggacaggggtctggggacaggggtgtggggacaggggtgtggggacaggggtctggggacaggggtgtggggacaggggtcctggggacaggggtgtggggacaggggtgtggggacaggggtgtggggacaggggtgtggggacaggggtcctggggataggggtgtggggacaggggtgtggggacaggggtcccggggacaggggtgtggggacaggggtgtggggacaggggtcctggggacaggggtctgaggacaggggtgtgggcacaggggtcctggggacaggggtcctggggacaggggtcctggggacaggggtctggggacagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccgccctaatgggccaggcggcaggggttgacaggtaggggagatgggctctgagactataaagccagcgggggcccagcagccctcagccctccaggacaggctgcatcagaagaggccatcaagcaggtctgttccaagggcctttgcgtcaggtgggctcagggttccagggtggctggaccccaggccccagctctgcagcagggaggacgtggctgggctcgtgaagcatgtgggggtgagcccaggggccccaaggcagggcacctggccttcagcctgcctcagccctgcctgtctcccagatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcagggtgagccaaccgcccattgctgcccctggccgcccccagccaccccctgctcctggcgctcccacccagcatgggcagaagggggcaggaggctgccacccagcagggggtcaggtgcacttttttaaaaagaagttctcttggtcacgtcctaaaagtgaccagctccctgtggcccagtcagaatctcagcctgaggacggtgttggcttcggcagccccgagatacatcagagggtgggcacgctcctccctccactcgcccctcaaacaaatgccccgcagcccatttctccaccctcatttgatgaccgcagattcaagtgttttgttaagtaaagtcctgggtgacctggggtcacagggtgccccacgctgcctgcctctgggcgaacaccccatcacgcccggaggagggcgtggctgcctgcctgagtgggccagacccctgtcgccagcctcacggcagctccatagtcaggagatggggaagatgctggggacaggccctggggagaagtactgggatcacctgttcaggctcccactgtgacgctgccccggggcgggggaaggaggtgggacatgtgggcgttggggcctgtaggtccacacccagtgtgggtgaccctccctctaacctgggtccagcccggctggagatgggtgggagtgcgacctagggctggcgggcaggcgggcactgtgtctccctgactgtgtcctcctgtgtccctctgcctcgccgctgttccggaacctgctctgcgcggcacgtcctggcagtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcctgcaggcagccccacacccgccgcctcctgcaccgagagagatggaataaagcccttgaaccagccctgctgtgccgtctgtgtgtcttgggggccctgggccaagccccacttcccggcactgttgtgagcccctcccagctctctccacgctctctgggtgcccacaggtgccaacgccaggcaggcccagcatgcagtggctctccccaaagcggccatgcctgttggctgcctgctgcccccaccctgtggctcagggtccagtatgggagcttcgggggtctctgaggggccagggatggtggggccactgagaagtgactctgtcagtagccgacctggagtccccagagaccttgttcaggaaagggaatgagaacattccagcaattttccccccacctagccctcccaggttctatttttagagttatttctgatggagtccctgtggagggaggaggctgggctgagggagggggtcctgcagggcggggggctgggaaggtggggagaggctgccgagagccacccgctatccccagctctgggcagccccgggacagtcacacaccctggcctcgcggcccaagctggcagccgtctgcagccacagcttatgccagcccaggtccagccagacacctgagggacccactggtgccttggaggaagcaggagaggtcagatggcaccatgagctggggcaggtgcagggaccgtggcagcacctggcagggcctcagaacccatgccttgggcaccccggccatgaggccctgaggattgcagcccaagagaagcagggaacgccagggccacaggggcagagaccaggccagggtcccttgcggcccttagcccaccccctcccagtaagcaggggctgcttggctaggcttccttttgctacagacctgctgctcacccagaggcccacgggccctagtgacaaggtcgttgtggctccaggtccttgggggtcctgacacagagcctcttctgcagcacccctgaggacagggtgctccgctgggcacccagcctagtgggcagacgagaacctaggggctgcctgggcctactgtggcctgggaggtcagcgggtgaccctagctaccctgtggctgggccagtctgcctgccacccaggccaaaccaatctgcacctttcctgagagctccacccagggctgggctggggatggctgggcctggggctggcatgggctgtggctgcagaccactgccagcttgggcctcgaggccaggagctcaccctccagctgccccgcctccagagtgggggccagggctgggcaggcgggtggacggccggacactggccccggaagaggagggaggcggtggctgggatcggcagcagccgtccatgggaacacccagccggccccactcgcacgggtagagacaggcgc